SEQUENCE LISTING (151 sequences)

SEQ ID NO: 1 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
ggcccagactgagcacgtgagttttagagctagaaatagcaagttaaaataaggctagtccgttatcaacttgaaaaagtgggaccgagtcggtcctctgccatcagaattccgtgctcagtctg

SEQ ID NO: 2 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
aattatggggattactaggagttttagagctagaaatagcaagttaaaataaggctagtccgttatcaacttgaaaaagtggcaccgagtcggtgctgccccttccgaattctagtaatccccat

SEQ ID NO: 3 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
aattatggggattactaggagttttagagctagaaatagcaagttaaaataaggctagtccgttatcaacttgaaaaagtggcaccgagtcggtgctgctgccccttccgaattctagtaatccccat

SEQ ID NO: 4 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
aattatggggattactaggagttttagagctagaaatagcaagttaaaataaggctagtccgttatcaacttgaaaaagtgggaccgagtcggtcctgccccttccgaattctagtaatccccat

SEQ ID NO: 5 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
aattatggggattactaggagttttagagctagaaatagcaagttaaaataaggctagtccgttatcaacttgaaaaagtgggaccgagtcggtcctgctgccccttccgaattctagtaatccccat

SEQ ID NO: 6 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
tccatttcatagtctttcctgttttagagctagaaatagcaagttaaaataaggctagtccgttatcaacttgaaaaagtggcaccgagtcggtgcacacccaagggaattcaaagactatgaaa

SEQ ID NO: 7 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
tccatttcatagtctttcctgttttagagctagaaatagcaagttaaaataaggctagtccgttatcaacttgaaaaagtggcaccgagtcggtgcaacacacccaagggaattcaaagactatgaaa

SEQ ID NO: 8 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
tccatttcatagtctttcctgttttagagctagaaatagcaagttaaaataaggctagtccgttatcaacttgaaaaagtgggaccgagtcggtccacacccaagggaattcaaagactatgaaa

SEQ ID NO: 9 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
tccatttcatagtctttcctgttttagagctagaaatagcaagttaaaataaggctagtccgttatcaacttgaaaaagtgggaccgagtcggtccaacacacccaagggaattcaaagactatgaaa

SEQ ID NO: 10 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
ggtcacttttaacacacccagttttagagctagaaatagcaagttaaaataaggctagtccgttatcaacttgaaaaagtggcaccgagtcggtgcctttccttgggaattcgtgtgttaaaagt

SEQ ID NO: 11 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
ggtcacttttaacacacccagttttagagctagaaatagcaagttaaaataaggctagtccgttatcaacttgaaaaagtggcaccgagtcggtgcagtctttccttgggaattcgtgtgttaaaagt

SEQ ID NO: 12 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
ggtcacttttaacacacccagttttagagctagaaatagcaagttaaaataaggctagtccgttatcaacttgaaaaagtgggaccgagtcggtccctttccttgggaattcgtgtgttaaaagt

SEQ ID NO: 13 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
ggtcacttttaacacacccagttttagagctagaaatagcaagttaaaataaggctagtccgttatcaacttgaaaaagtgggaccgagtcggtccagtctttccttgggaattcgtgtgttaaaagt

SEQ ID NO: 14 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
ttttgtaattaacagcttgcgttttagagctagaaatagcaagttaaaataaggctagtccgttatcaacttgaaaaagtggcaccgagtcggtgcttcaccagcagaattcagctgttaattac

SEQ ID NO: 15 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
ttttgtaattaacagcttgcgttttagagctagaaatagcaagttaaaataaggctagtccgttatcaacttgaaaaagtggcaccgagtcggtgccttttcaccagcagaattcagctgttaattac

SEQ ID NO: 16 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
ttttgtaattaacagcttgcgttttagagctagaaatagcaagttaaaataaggctagtccgttatcaacttgaaaaagtgggaccgagtcggtccttcaccagcagaattcagctgttaattac

SEQ ID NO: 17 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
ttttgtaattaacagcttgcgttttagagctagaaatagcaagttaaaataaggctagtccgttatcaacttgaaaaagtgggaccgagtcggtcccttttcaccagcagaattcagctgttaattac

SEQ ID NO: 18 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
cttatatccaacacttcgtggttttagagctagaaatagcaagttaaaataaggctagtccgttatcaacttgaaaaagtggcaccgagtcggtgcaggaccccacgaattcgaagtgttggata

SEQ ID NO: 19 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
cttatatccaacacttcgtggttttagagctagaaatagcaagttaaaataaggctagtccgttatcaacttgaaaaagtggcaccgagtcggtgcaaaaggaccccacgaattcgaagtgttggata

SEQ ID NO: 20 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
cttatatccaacacttcgtggttttagagctagaaatagcaagttaaaataaggctagtccgttatcaacttgaaaaagtgggaccgagtcggtccaggaccccacgaattcgaagtgttggata

SEQ ID NO: 21 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
cttatatccaacacttcgtggttttagagctagaaatagcaagttaaaataaggctagtccgttatcaacttgaaaaagtgggaccgagtcggtccaaaaggaccccacgaattcgaagtgttggata

SEQ ID NO: 22 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
ggcttatatccaacacttcggttttagagctagaaatagcaagttaaaataaggctagtccgttatcaacttgaaaaagtggcaccgagtcggtgcgaccccacgagaattcagtgttggatata

SEQ ID NO: 23 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
ggcttatatccaacacttcggttttagagctagaaatagcaagttaaaataaggctagtccgttatcaacttgaaaaagtggcaccgagtcggtgcaaggaccccacgagaattcagtgttggatata

SEQ ID NO: 24 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
ggcttatatccaacacttcggttttagagctagaaatagcaagttaaaataaggctagtccgttatcaacttgaaaaagtgggaccgagtcggtccgaccccacgagaattcagtgttggatata

SEQ ID NO: 25 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
ggcttatatccaacacttcggttttagagctagaaatagcaagttaaaataaggctagtccgttatcaacttgaaaaagtgggaccgagtcggtccaaggaccccacgagaattcagtgttggatata

SEQ ID NO: 26 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
atttcacataaaactcttttgttttagagctagaaatagcaagttaaaataaggctagtccgttatcaacttgaaaaagtggcaccgagtcggtgcttaacctaaagaattcagagttttatgtg

SEQ ID NO: 27 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
atttcacataaaactcttttgttttagagctagaaatagcaagttaaaataaggctagtccgttatcaacttgaaaaagtggcaccgagtcggtgctctttaacctaaagaattcagagttttatgtg

SEQ ID NO: 28 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
atttcacataaaactcttttgttttagagctagaaatagcaagttaaaataaggctagtccgttatcaacttgaaaaagtgggaccgagtcggtccttaacctaaagaattcagagttttatgtg

SEQ ID NO: 29 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
atttcacataaaactcttttgttttagagctagaaatagcaagttaaaataaggctagtccgttatcaacttgaaaaagtgggaccgagtcggtcctctttaacctaaagaattcagagttttatgtg

SEQ ID NO: 30 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
cttagagaatatttgtagaggttttagagctagaaatagcaagttaaaataaggctagtccgttatcaacttgaaaaagtggcaccgagtcggtgctgtgcctctcgaattctacaaatattctc

SEQ ID NO: 31 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
cttagagaatatttgtagaggttttagagctagaaatagcaagttaaaataaggctagtccgttatcaacttgaaaaagtggcaccgagtcggtgcaaatgtgcctctcgaattctacaaatattctc

SEQ ID NO: 32 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
cttagagaatatttgtagaggttttagagctagaaatagcaagttaaaataaggctagtccgttatcaacttgaaaaagtgggaccgagtcggtcctgtgcctctcgaattctacaaatattctc

SEQ ID NO: 33 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
cttagagaatatttgtagaggttttagagctagaaatagcaagttaaaataaggctagtccgttatcaacttgaaaaagtgggaccgagtcggtccaaatgtgcctctcgaattctacaaatattctc

SEQ ID NO: 34 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
ttgactataatgaatacttcgttttagagctagaaatagcaagttaaaataaggctagtccgttatcaacttgaaaaagtggcaccgagtcggtgcaatccctgaagaattcgtattcattatag

SEQ ID NO: 35 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
ttgactataatgaatacttcgttttagagctagaaatagcaagttaaaataaggctagtccgttatcaacttgaaaaagtggcaccgagtcggtgctcaaatccctgaagaattcgtattcattatag

SEQ ID NO: 36 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
ttgactataatgaatacttcgttttagagctagaaatagcaagttaaaataaggctagtccgttatcaacttgaaaaagtgggaccgagtcggtccaatccctgaagaattcgtattcattatag

SEQ ID NO: 37 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
ttgactataatgaatacttcgttttagagctagaaatagcaagttaaaataaggctagtccgttatcaacttgaaaaagtgggaccgagtcggtcctcaaatccctgaagaattcgtattcattatag

SEQ ID NO: 38 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
caaaacacgcataaaaatttgttttagagctagaaatagcaagttaaaataaggctagtccgttatcaacttgaaaaagtggcaccgagtcggtgcctttcctaaagaattctttttatgcgtgt

SEQ ID NO: 39 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
caaaacacgcataaaaatttgttttagagctagaaatagcaagttaaaataaggctagtccgttatcaacttgaaaaagtggcaccgagtcggtgctctctttcctaaagaattctttttatgcgtgt

SEQ ID NO: 40 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
caaaacacgcataaaaatttgttttagagctagaaatagcaagttaaaataaggctagtccgttatcaacttgaaaaagtgggaccgagtcggtccctttcctaaagaattctttttatgcgtgt

SEQ ID NO: 41 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
caaaacacgcataaaaatttgttttagagctagaaatagcaagttaaaataaggctagtccgttatcaacttgaaaaagtgggaccgagtcggtcctctctttcctaaagaattctttttatgcgtgt

SEQ ID NO: 42 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
tcaaattatgaggtgctggagttttagagctagaaatagcaagttaaaataaggctagtccgttatcaacttgaaaaagtggcaccgagtcggtgcttctccttccgaattcagcacctcataat

SEQ ID NO: 43 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
tcaaattatgaggtgctggagttttagagctagaaatagcaagttaaaataaggctagtccgttatcaacttgaaaaagtggcaccgagtcggtgcgttttctccttccgaattcagcacctcataat

SEQ ID NO: 44 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
tcaaattatgaggtgctggagttttagagctagaaatagcaagttaaaataaggctagtccgttatcaacttgaaaaagtgggaccgagtcggtccttctccttccgaattcagcacctcataat

SEQ ID NO: 45 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
tcaaattatgaggtgctggagttttagagctagaaatagcaagttaaaataaggctagtccgttatcaacttgaaaaagtgggaccgagtcggtccgttttctccttccgaattcagcacctcataat

SEQ ID NO: 46 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
tacagctttatgtgactaatgttttagagctagaaatagcaagttaaaataaggctagtccgttatcaacttgaaaaagtggcaccgagtcggtgcggttcccattgaattcagtcacataaagc

SEQ ID NO: 47 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
tacagctttatgtgactaatgttttagagctagaaatagcaagttaaaataaggctagtccgttatcaacttgaaaaagtggcaccgagtcggtgcgatggttcccattgaattcagtcacataaagc

SEQ ID NO: 48 - RNA, 125 nt, synthetic construct
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond
tacagctttatgtgactaatgttttagagctagaaatagcaagttaaaataaggctagtccgttatcaacttgaaaaagtgggaccgagtcggtccggttcccattgaattcagtcacataaagc

SEQ ID NO: 49 - RNA, 128 nt, synthetic construct
  source 1..128: mol_type = other RNA
  modified_base order(1..3,126..128): mod_base = OTHER; note = 2-Prime-O-methyl ribonucleotides
  modified_base order(1..4,125..128): mod_base = OTHER; note = Phosphorothioate bond
tacagctttatgtgactaatgttttagagctagaaatagcaagttaaaataaggctagtccgttatcaacttgaaaaagtgggaccgagtcggtccgatggttcccattgaattcagtcacataaagc

SEQ ID NO: 50 - RNA, 125 nt, synthetic construct; identical to SEQ ID NO: 46
  source 1..125: mol_type = other RNA
  modified_base 1..3: mod_base = OTHER; note = 2-Prime-O-methyl ribonucleotides
  modified_base 1..4: mod_base = OTHER; note = Phosphorothioate bond

SEQ ID NO: 51 - RNA, 125 nt, synthetic construct; identical to SEQ ID NO: 46
  source 1..125: mol_type = other RNA
  modified_base order(1..3,125): mod_base = OTHER; note = 2-Prime-O-methyl ribonucleotides
  modified_base 1..4: mod_base = OTHER; note = Phosphorothioate bond

SEQ ID NO: 52 - RNA, 125 nt, synthetic construct; identical to SEQ ID NO: 46
  source 1..125: mol_type = other RNA
  modified_base order(1..3,124..125): mod_base = OTHER; note = 2-Prime-O-methyl ribonucleotides
  modified_base 1..4: mod_base = OTHER; note = Phosphorothioate bond

SEQ ID NO: 53 - RNA, 125 nt, synthetic construct; identical to SEQ ID NO: 46
  source 1..125: mol_type = other RNA
  modified_base order(1..3,123..125): mod_base = OTHER; note = 2-Prime-O-methyl ribonucleotides
  modified_base 1..4: mod_base = OTHER; note = Phosphorothioate bond

SEQ ID NO: 54 - RNA, 125 nt, synthetic construct; identical to SEQ ID NO: 46
  source 1..125: mol_type = other RNA
  modified_base order(1..3,121..125): mod_base = OTHER; note = 2-Prime-O-methyl ribonucleotides
  modified_base 1..4: mod_base = OTHER; note = Phosphorothioate bond

SEQ ID NO: 55 - RNA, 125 nt, synthetic construct; identical to SEQ ID NO: 46
  source 1..125: mol_type = other RNA
  modified_base order(1..3,121..125): mod_base = OTHER; note = 2-Prime-O-methyl ribonucleotides
  modified_base 1..4: mod_base = OTHER; note = Phosphorothioate bond

SEQ ID NO: 56 - RNA, 125 nt, synthetic construct; identical to SEQ ID NO: 46
  source 1..125: mol_type = other RNA
  modified_base order(1..3,121..125): mod_base = OTHER; note = 2-Prime-O-methyl ribonucleotides
  modified_base 1..4: mod_base = OTHER; note = Phosphorothioate bond

SEQ ID NO: 57 - RNA, 125 nt, synthetic construct; identical to SEQ ID NO: 46
  source 1..125: mol_type = other RNA
  modified_base order(1..3,119..125): mod_base = OTHER; note = 2-Prime-O-methyl ribonucleotides
  modified_base 1..4: mod_base = OTHER; note = Phosphorothioate bond

SEQ ID NO: 58 - RNA, 125 nt, synthetic construct; identical to SEQ ID NO: 46
  source 1..125: mol_type = other RNA
  modified_base order(1..3,118..125): mod_base = OTHER; note = 2-Prime-O-methyl ribonucleotides
  modified_base 1..4: mod_base = OTHER; note = Phosphorothioate bond

SEQ ID NO: 59 - RNA, 125 nt, synthetic construct; identical to SEQ ID NO: 46
  source 1..125: mol_type = other RNA
  modified_base order(1..3,117..125): mod_base = OTHER; note = 2-Prime-O-methyl ribonucleotides
  modified_base 1..4: mod_base = OTHER; note = Phosphorothioate bond

SEQ ID NO: 60 - RNA, 125 nt, synthetic construct; identical to SEQ ID NO: 46
  source 1..125: mol_type = other RNA
  modified_base order(1..3,116..125): mod_base = OTHER; note = 2-Prime-O-methyl ribonucleotides
  modified_base 1..4: mod_base = OTHER; note = Phosphorothioate bond

SEQ ID NO: 61 - RNA, 125 nt, synthetic construct; identical to SEQ ID NO: 46
  source 1..125: mol_type = other RNA
  modified_base 1..3: mod_base = OTHER; note = 2-Prime-O-methyl ribonucleotides
  modified_base order(1..4,124..125): mod_base = OTHER; note = Phosphorothioate bond

SEQ ID NO: 62 - RNA, 125 nt, synthetic construct; identical to SEQ ID NO: 46
  source 1..125: mol_type = other RNA
  modified_base 1..3: mod_base = OTHER; note = 2-Prime-O-methyl ribonucleotides
  modified_base order(1..4,123..125): mod_base = OTHER; note = Phosphorothioate bond

SEQ ID NO: 63 - RNA, 125 nt, synthetic construct; identical to SEQ ID NO: 46
  source 1..125: mol_type = other RNA
  modified_base 1..3: mod_base = OTHER; note = 2-Prime-O-methyl ribonucleotides
  modified_base order(1..4,122..125): mod_base = OTHER; note = Phosphorothioate bond

SEQ ID NO: 64 - RNA, 125 nt, synthetic construct; identical to SEQ ID NO: 46
  source 1..125: mol_type = other RNA
  modified_base 1..3: mod_base = OTHER; note = 2-Prime-O-methyl ribonucleotides
  modified_base order(1..4,122..125): mod_base = OTHER; note = Phosphorothioate bond

SEQ ID NO: 65 - RNA, 125 nt, synthetic construct; identical to SEQ ID NO: 46
  source 1..125: mol_type = other RNA
  modified_base 1..3: mod_base = OTHER; note = 2-Prime-O-methyl ribonucleotides
  modified_base order(1..4,120..125): mod_base = OTHER; note = Phosphorothioate bond

SEQ ID NO: 66 - RNA, 125 nt, synthetic construct; identical to SEQ ID NO: 46
  source 1..125: mol_type = other RNA
  modified_base 1..3: mod_base = OTHER; note = 2-Prime-O-methyl ribonucleotides
  modified_base order(1..4,119..125): mod_base = OTHER; note = Phosphorothioate bond

SEQ ID NO: 67 - RNA, 125 nt, synthetic construct; identical to SEQ ID NO: 46
  source 1..125: mol_type = other RNA
  modified_base 1..3: mod_base = OTHER; note = 2-Prime-O-methyl ribonucleotides
  modified_base order(1..4,118..125): mod_base = OTHER; note = Phosphorothioate bond

SEQ ID NO: 68 - RNA, 125 nt, synthetic construct; identical to SEQ ID NO: 46
  source 1..125: mol_type = other RNA
  modified_base 1..3: mod_base = OTHER; note = 2-Prime-O-methyl ribonucleotides
  modified_base order(1..4,117..125): mod_base = OTHER; note = Phosphorothioate bond

SEQ ID NO: 69 - RNA, 125 nt, synthetic construct; identical to SEQ ID NO: 46
  source 1..125: mol_type = other RNA
  modified_base 1..3: mod_base = OTHER; note = 2-Prime-O-methyl ribonucleotides
  modified_base order(1..4,116..125): mod_base = OTHER; note = Phosphorothioate bond

SEQ ID NO: 70 - RNA, 125 nt, synthetic construct; identical to SEQ ID NO: 46
  source 1..125: mol_type = other RNA
  modified_base 1..3: mod_base = OTHER; note = 2-Prime-O-methyl ribonucleotides
  modified_base order(1..4,115..125): mod_base = OTHER; note = Phosphorothioate bond

SEQ ID NO: 71 - RNA, 125 nt, synthetic construct; identical to SEQ ID NO: 46
  source 1..125: mol_type = other RNA
  modified_base order(1..3,125): mod_base = OTHER; note = 2-Prime-O-methyl ribonucleotides
  modified_base order(1..4,124..125): mod_base = OTHER; note = Phosphorothioate bond

SEQ ID NO: 72 - RNA, 125 nt, synthetic construct; identical to SEQ ID NO: 46
  source 1..125: mol_type = other RNA
  modified_base order(1..3,124..125): mod_base = OTHER; note = 2-Prime-O-methyl ribonucleotides
  modified_base order(1..4,123..125): mod_base = OTHER; note = Phosphorothioate bond

SEQ ID NO: 73 - RNA, 125 nt, synthetic construct; identical to SEQ ID NO: 46
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond

SEQ ID NO: 74 - RNA, 125 nt, synthetic construct; identical to SEQ ID NO: 46
  source 1..125: mol_type = other RNA
  modified_base order(1..3,122..125): mod_base = OTHER; note = 2-Prime-O-methyl ribonucleotides
  modified_base order(1..4,121..125): mod_base = OTHER; note = Phosphorothioate bond

SEQ ID NO: 75 - RNA, 125 nt, synthetic construct; identical to SEQ ID NO: 46
  source 1..125: mol_type = other RNA
  modified_base order(1..3,121..125): mod_base = OTHER; note = 2-Prime-O-methyl ribonucleotides
  modified_base order(1..4,120..125): mod_base = OTHER; note = Phosphorothioate bond

SEQ ID NO: 76 - RNA, 125 nt, synthetic construct; identical to SEQ ID NO: 46
  source 1..125: mol_type = other RNA
  modified_base order(1..3,120..125): mod_base = OTHER; note = 2-Prime-O-methyl ribonucleotides
  modified_base order(1..4,119..125): mod_base = OTHER; note = Phosphorothioate bond

SEQ ID NO: 77 - RNA, 125 nt, synthetic construct; identical to SEQ ID NO: 46
  source 1..125: mol_type = other RNA
  modified_base order(1..3,119..125): mod_base = OTHER; note = 2-Prime-O-methyl ribonucleotides
  modified_base order(1..4,118..125): mod_base = OTHER; note = Phosphorothioate bond

SEQ ID NO: 78 - RNA, 125 nt, synthetic construct; identical to SEQ ID NO: 46
  source 1..125: mol_type = other RNA
  modified_base order(1..3,118..125): mod_base = OTHER; note = 2-Prime-O-methyl ribonucleotides
  modified_base order(1..4,117..125): mod_base = OTHER; note = Phosphorothioate bond

SEQ ID NO: 79 - RNA, 125 nt, synthetic construct; identical to SEQ ID NO: 46
  source 1..125: mol_type = other RNA
  modified_base order(1..3,117..125): mod_base = OTHER; note = 2-Prime-O-methyl ribonucleotides
  modified_base order(1..4,116..125): mod_base = OTHER; note = Phosphorothioate bond

SEQ ID NO: 80 - RNA, 125 nt, synthetic construct; identical to SEQ ID NO: 46
  source 1..125: mol_type = other RNA
  modified_base order(1..3,116..125): mod_base = OTHER; note = 2-Prime-O-methyl ribonucleotides
  modified_base order(1..4,115..125): mod_base = OTHER; note = Phosphorothioate bond

SEQ ID NO: 81 - RNA, 125 nt, synthetic construct; identical to SEQ ID NO: 46
  source 1..125: mol_type = other RNA
  modified_base order(1..3,122..124): mod_base = OTHER; note = 2-Prime-O-methyl ribonucleotides
  modified_base order(1..4,122..125): mod_base = OTHER; note = Phosphorothioate bond

SEQ ID NO: 82 - RNA, 125 nt, synthetic construct; identical to SEQ ID NO: 46
  source 1..125: mol_type = other RNA
  modified_base 123..125: mod_base = OTHER; note = 2-Prime-O-methyl ribonucleotides
  modified_base 122..125: mod_base = OTHER; note = Phosphorothioate bond

SEQ ID NO: 83 - RNA, 125 nt, synthetic construct; identical to SEQ ID NO: 46
  source 1..125: mol_type = other RNA
  modified_base order(1,123..125): mod_base = OTHER; note = 2-Prime-O-methyl ribonucleotides
  modified_base 122..125: mod_base = OTHER; note = Phosphorothioate bond

SEQ ID NO: 84 - RNA, 125 nt, synthetic construct; identical to SEQ ID NO: 46
  source 1..125: mol_type = other RNA
  modified_base order(1..2,123..125): mod_base = OTHER; note = 2-Prime-O-methyl ribonucleotides
  modified_base 122..125: mod_base = OTHER; note = Phosphorothioate bond

SEQ ID NO: 85 - RNA, 125 nt, synthetic construct; identical to SEQ ID NO: 46
  source 1..125: mol_type = other RNA
  modified_base order(1..4,123..125): mod_base = OTHER; note = 2-Prime-O-methyl ribonucleotides
  modified_base 122..125: mod_base = OTHER; note = Phosphorothioate bond

SEQ ID NO: 86 - RNA, 125 nt, synthetic construct; identical to SEQ ID NO: 46
  source 1..125: mol_type = other RNA
  modified_base order(1..5,123..125): mod_base = OTHER; note = 2-Prime-O-methyl ribonucleotides
  modified_base 122..125: mod_base = OTHER; note = Phosphorothioate bond

SEQ ID NO: 87 - RNA, 125 nt, synthetic construct; identical to SEQ ID NO: 46
  source 1..125: mol_type = other RNA
  modified_base order(1..6,123..125): mod_base = OTHER; note = 2-Prime-O-methyl ribonucleotides
  modified_base 122..125: mod_base = OTHER; note = Phosphorothioate bond

SEQ ID NO: 88 - RNA, 125 nt, synthetic construct; identical to SEQ ID NO: 46
  source 1..125: mol_type = other RNA
  modified_base order(1..7,123..125): mod_base = OTHER; note = 2-Prime-O-methyl ribonucleotides
  modified_base 122..125: mod_base = OTHER; note = Phosphorothioate bond

SEQ ID NO: 89 - RNA, 125 nt, synthetic construct; identical to SEQ ID NO: 46
  source 1..125: mol_type = other RNA
  modified_base order(1..8,123..125): mod_base = OTHER; note = 2-Prime-O-methyl ribonucleotides
  modified_base 122..125: mod_base = OTHER; note = Phosphorothioate bond

SEQ ID NO: 90 - RNA, 125 nt, synthetic construct; identical to SEQ ID NO: 46
  source 1..125: mol_type = other RNA
  modified_base order(1..9,123..125): mod_base = OTHER; note = 2-Prime-O-methyl ribonucleotides
  modified_base 122..125: mod_base = OTHER; note = Phosphorothioate bond

SEQ ID NO: 91 - RNA, 125 nt, synthetic construct; identical to SEQ ID NO: 46
  source 1..125: mol_type = other RNA
  modified_base order(1..10,123..125): mod_base = OTHER; note = 2-Prime-O-methyl ribonucleotides
  modified_base 122..125: mod_base = OTHER; note = Phosphorothioate bond

SEQ ID NO: 92 - RNA, 125 nt, synthetic construct; identical to SEQ ID NO: 46
  source 1..125: mol_type = other RNA
  modified_base 123..125: mod_base = OTHER; note = 2-Prime-O-methyl ribonucleotides
  modified_base order(1..2,122..125): mod_base = OTHER; note = Phosphorothioate bond

SEQ ID NO: 93 - RNA, 125 nt, synthetic construct; identical to SEQ ID NO: 46
  source 1..125: mol_type = other RNA
  modified_base 123..125: mod_base = OTHER; note = 2-Prime-O-methyl ribonucleotides
  modified_base order(1..3,122..125): mod_base = OTHER; note = Phosphorothioate bond

SEQ ID NO: 94 - RNA, 125 nt, synthetic construct; identical to SEQ ID NO: 46
  source 1..125: mol_type = other RNA
  modified_base 123..125: mod_base = OTHER; note = 2-Prime-O-methyl ribonucleotides
  modified_base order(1..4,122..125): mod_base = OTHER; note = Phosphorothioate bond

SEQ ID NO: 95 - RNA, 125 nt, synthetic construct; identical to SEQ ID NO: 46
  source 1..125: mol_type = other RNA
  modified_base 123..125: mod_base = OTHER; note = 2-Prime-O-methyl ribonucleotides
  modified_base order(1..5,122..125): mod_base = OTHER; note = Phosphorothioate bond

SEQ ID NO: 96 - RNA, 125 nt, synthetic construct; identical to SEQ ID NO: 46
  source 1..125: mol_type = other RNA
  modified_base 123..125: mod_base = OTHER; note = 2-Prime-O-methyl ribonucleotides
  modified_base order(1..6,122..125): mod_base = OTHER; note = Phosphorothioate bond

SEQ ID NO: 97 - RNA, 125 nt, synthetic construct; identical to SEQ ID NO: 46
  source 1..125: mol_type = other RNA
  modified_base 123..125: mod_base = OTHER; note = 2-Prime-O-methyl ribonucleotides
  modified_base order(1..7,122..125): mod_base = OTHER; note = Phosphorothioate bond

SEQ ID NO: 98 - RNA, 125 nt, synthetic construct; identical to SEQ ID NO: 46
  source 1..125: mol_type = other RNA
  modified_base 123..125: mod_base = OTHER; note = 2-Prime-O-methyl ribonucleotides
  modified_base order(1..8,122..125): mod_base = OTHER; note = Phosphorothioate bond

SEQ ID NO: 99 - RNA, 125 nt, synthetic construct; identical to SEQ ID NO: 46
  source 1..125: mol_type = other RNA
  modified_base 123..125: mod_base = OTHER; note = 2-Prime-O-methyl ribonucleotides
  modified_base order(1..9,122..125): mod_base = OTHER; note = Phosphorothioate bond

SEQ ID NO: 100 - RNA, 125 nt, synthetic construct; identical to SEQ ID NO: 46
  source 1..125: mol_type = other RNA
  modified_base 123..125: mod_base = OTHER; note = 2-Prime-O-methyl ribonucleotides
  modified_base order(1..10,122..125): mod_base = OTHER; note = Phosphorothioate bond

SEQ ID NO: 101 - RNA, 125 nt, synthetic construct; identical to SEQ ID NO: 46
  source 1..125: mol_type = other RNA
  modified_base 123..125: mod_base = OTHER; note = 2-Prime-O-methyl ribonucleotides
  modified_base order(1..11,122..125): mod_base = OTHER; note = Phosphorothioate bond

SEQ ID NO: 102 - RNA, 125 nt, synthetic construct; identical to SEQ ID NO: 46
  source 1..125: mol_type = other RNA
  modified_base order(1,123..125): mod_base = OTHER; note = 2-Prime-O-methyl ribonucleotides
  modified_base order(1..2,122..125): mod_base = OTHER; note = Phosphorothioate bond

SEQ ID NO: 103 - RNA, 125 nt, synthetic construct; identical to SEQ ID NO: 46
  source 1..125: mol_type = other RNA
  modified_base order(1..2,123..125): mod_base = OTHER; note = 2-Prime-O-methyl ribonucleotides
  modified_base order(1..3,122..125): mod_base = OTHER; note = Phosphorothioate bond

SEQ ID NO: 104 - RNA, 125 nt, synthetic construct; identical to SEQ ID NO: 46
  source 1..125: mol_type = other RNA
  modified_base order(1..3,123..125): mod_base = OTHER; note = 2-Prime-O-methyl ribonucleotides
  modified_base order(1..4,122..125): mod_base = OTHER; note = Phosphorothioate bond

SEQ ID NO: 105 - RNA, 125 nt, synthetic construct; identical to SEQ ID NO: 46
  source 1..125: mol_type = other RNA
  modified_base order(1..4,123..125): mod_base = OTHER; note = 2-Prime-O-methyl ribonucleotides
  modified_base order(1..5,122..125): mod_base = OTHER; note = Phosphorothioate bond

SEQ ID NO: 106 - RNA, 125 nt, synthetic construct; identical to SEQ ID NO: 46
  source 1..125: mol_type = other RNA
  modified_base order(1..5,123..125): mod_base = OTHER; note = 2-Prime-O-methyl ribonucleotides
  modified_base order(1..6,122..125): mod_base = OTHER; note = Phosphorothioate bond

SEQ ID NO: 107 - RNA, 125 nt, synthetic construct; identical to SEQ ID NO: 46
  source 1..125: mol_type = other RNA
  modified_base order(1..6,123..125): mod_base = OTHER; note = 2-Prime-O-methyl ribonucleotides
  modified_base order(1..7,122..125): mod_base = OTHER; note = Phosphorothioate bond

SEQ ID NO: 108 - RNA, 125 nt, synthetic construct; identical to SEQ ID NO: 46
  source 1..125: mol_type = other RNA
  modified_base order(1..7,123..125): mod_base = OTHER; note = 2-Prime-O-methyl ribonucleotides
  modified_base order(1..8,122..125): mod_base = OTHER; note = Phosphorothioate bond

SEQ ID NO: 109 - RNA, 125 nt, synthetic construct; identical to SEQ ID NO: 46
  source 1..125: mol_type = other RNA
  modified_base order(1..8,123..125): mod_base = OTHER; note = 2-Prime-O-methyl ribonucleotides
  modified_base order(1..9,122..125): mod_base = OTHER; note = Phosphorothioate bond

SEQ ID NO: 110 - RNA, 125 nt, synthetic construct; identical to SEQ ID NO: 46
  source 1..125: mol_type = other RNA
  modified_base order(1..9,123..125): mod_base = OTHER; note = 2-Prime-O-methyl ribonucleotides
  modified_base order(1..10,122..125): mod_base = OTHER; note = Phosphorothioate bond

SEQ ID NO: 111 - RNA, 125 nt, synthetic construct; identical to SEQ ID NO: 46
  source 1..125: mol_type = other RNA
  modified_base order(1..10,123..125): mod_base = OTHER; note = 2-Prime-O-methyl ribonucleotides
  modified_base order(1..11,122..125): mod_base = OTHER; note = Phosphorothioate bond

SEQ ID NO: 112 - RNA, 125 nt, synthetic construct; identical to SEQ ID NO: 46
  source 1..125: mol_type = other RNA
  modified_base order(1..3,113..125): mod_base = OTHER; note = 2-Prime-O-methyl ribonucleotides
  modified_base order(1..4,122..125): mod_base = OTHER; note = Phosphorothioate bond

SEQ ID NO: 113 - RNA, 125 nt, synthetic construct; identical to SEQ ID NO: 46
  source 1..125: mol_type = other RNA
  modified_base order(1..3,113..125): mod_base = OTHER; note = 2-Prime-O-methyl ribonucleotides
  modified_base order(1..4,112..125): mod_base = OTHER; note = Phosphorothioate bond

SEQ ID NO: 114 - RNA, 125 nt, synthetic construct; identical to SEQ ID NO: 2
  source 1..125: mol_type = other RNA
  modified_base order(1..3,119..125): mod_base = OTHER; note = 2-Prime-O-methyl ribonucleotides
  modified_base order(1..4,118..125): mod_base = OTHER; note = Phosphorothioate bond

SEQ ID NO: 115 - RNA, 125 nt, synthetic construct; identical to SEQ ID NO: 6
  source 1..125: mol_type = other RNA
  modified_base order(1..3,119..125): mod_base = OTHER; note = 2-Prime-O-methyl ribonucleotides
  modified_base order(1..4,118..125): mod_base = OTHER; note = Phosphorothioate bond

SEQ ID NO: 116 - RNA, 125 nt, synthetic construct; identical to SEQ ID NO: 10
  source 1..125: mol_type = other RNA
  modified_base order(1..3,119..125): mod_base = OTHER; note = 2-Prime-O-methyl ribonucleotides
  modified_base order(1..4,118..125): mod_base = OTHER; note = Phosphorothioate bond

SEQ ID NO: 117 - RNA, 125 nt, synthetic construct; identical to SEQ ID NO: 14
  source 1..125: mol_type = other RNA
  modified_base order(1..3,119..125): mod_base = OTHER; note = 2-Prime-O-methyl ribonucleotides
  modified_base order(1..4,118..125): mod_base = OTHER; note = Phosphorothioate bond

SEQ ID NO: 118 - RNA, 125 nt, synthetic construct; identical to SEQ ID NO: 18
  source 1..125: mol_type = other RNA
  modified_base order(1..3,119..125): mod_base = OTHER; note = 2-Prime-O-methyl ribonucleotides
  modified_base order(1..4,118..125): mod_base = OTHER; note = Phosphorothioate bond

SEQ ID NO: 119 - RNA, 125 nt, synthetic construct; identical to SEQ ID NO: 22
  source 1..125: mol_type = other RNA
  modified_base order(1..3,119..125): mod_base = OTHER; note = 2-Prime-O-methyl ribonucleotides
  modified_base order(1..4,118..125): mod_base = OTHER; note = Phosphorothioate bond

SEQ ID NO: 120 - RNA, 125 nt, synthetic construct; identical to SEQ ID NO: 26
  source 1..125: mol_type = other RNA
  modified_base order(1..3,119..125): mod_base = OTHER; note = 2-Prime-O-methyl ribonucleotides
  modified_base order(1..4,118..125): mod_base = OTHER; note = Phosphorothioate bond

SEQ ID NO: 121 - RNA, 125 nt, synthetic construct; identical to SEQ ID NO: 30
  source 1..125: mol_type = other RNA
  modified_base order(1..3,119..125): mod_base = OTHER; note = 2-Prime-O-methyl ribonucleotides
  modified_base order(1..4,118..125): mod_base = OTHER; note = Phosphorothioate bond

SEQ ID NO: 122 - RNA, 125 nt, synthetic construct; identical to SEQ ID NO: 34
  source 1..125: mol_type = other RNA
  modified_base order(1..3,119..125): mod_base = OTHER; note = 2-Prime-O-methyl ribonucleotides
  modified_base order(1..4,118..125): mod_base = OTHER; note = Phosphorothioate bond

SEQ ID NO: 123 - RNA, 125 nt, synthetic construct; identical to SEQ ID NO: 38
  source 1..125: mol_type = other RNA
  modified_base order(1..3,119..125): mod_base = OTHER; note = 2-Prime-O-methyl ribonucleotides
  modified_base order(1..4,118..125): mod_base = OTHER; note = Phosphorothioate bond

SEQ ID NO: 124 - RNA, 125 nt, synthetic construct; identical to SEQ ID NO: 42
  source 1..125: mol_type = other RNA
  modified_base order(1..3,119..125): mod_base = OTHER; note = 2-Prime-O-methyl ribonucleotides
  modified_base order(1..4,118..125): mod_base = OTHER; note = Phosphorothioate bond

SEQ ID NO: 125 - RNA, 125 nt, synthetic construct; identical to SEQ ID NO: 46
  source 1..125: mol_type = other RNA
  modified_base order(1..3,119..125): mod_base = OTHER; note = 2-Prime-O-methyl ribonucleotides
  modified_base order(1..4,118..125): mod_base = OTHER; note = Phosphorothioate bond

SEQ ID NO: 126 - RNA, 125 nt, synthetic construct; identical to SEQ ID NO: 2
  source 1..125: mol_type = other RNA
  modified_base order(1..2,119..125): mod_base = OTHER; note = 2-Prime-O-methyl ribonucleotides
  modified_base order(1..3,118..125): mod_base = OTHER; note = Phosphorothioate bond

SEQ ID NO: 127 - RNA, 125 nt, synthetic construct; identical to SEQ ID NO: 6
  source 1..125: mol_type = other RNA
  modified_base order(1..2,119..125): mod_base = OTHER; note = 2-Prime-O-methyl ribonucleotides
  modified_base order(1..3,118..125): mod_base = OTHER; note = Phosphorothioate bond

SEQ ID NO: 128 - RNA, 125 nt, synthetic construct; identical to SEQ ID NO: 10
  source 1..125: mol_type = other RNA
  modified_base order(1..2,119..125): mod_base = OTHER; note = 2-Prime-O-methyl ribonucleotides
  modified_base order(1..3,118..125): mod_base = OTHER; note = Phosphorothioate bond

SEQ ID NO: 129 - RNA, 125 nt, synthetic construct; identical to SEQ ID NO: 14
  source 1..125: mol_type = other RNA
  modified_base order(1..2,119..125): mod_base = OTHER; note = 2-Prime-O-methyl ribonucleotides
  modified_base order(1..3,118..125): mod_base = OTHER; note = Phosphorothioate bond

SEQ ID NO: 130 - RNA, 125 nt, synthetic construct; identical to SEQ ID NO: 18
  source 1..125: mol_type = other RNA
  modified_base order(1..2,119..125): mod_base = OTHER; note = 2-Prime-O-methyl ribonucleotides
  modified_base order(1..3,118..125): mod_base = OTHER; note = Phosphorothioate bond

SEQ ID NO: 131 - RNA, 125 nt, synthetic construct; identical to SEQ ID NO: 22
  source 1..125: mol_type = other RNA
  modified_base order(1..2,119..125): mod_base = OTHER; note = 2-Prime-O-methyl ribonucleotides
  modified_base order(1..3,118..125): mod_base = OTHER; note = Phosphorothioate bond

SEQ ID NO: 132 - RNA, 125 nt, synthetic construct; identical to SEQ ID NO: 26
  source 1..125: mol_type = other RNA
  modified_base order(1..2,119..125): mod_base = OTHER; note = 2-Prime-O-methyl ribonucleotides
  modified_base order(1..3,118..125): mod_base = OTHER; note = Phosphorothioate bond

SEQ ID NO: 133 - RNA, 125 nt, synthetic construct; identical to SEQ ID NO: 30
  source 1..125: mol_type = other RNA
  modified_base order(1..2,119..125): mod_base = OTHER; note = 2-Prime-O-methyl ribonucleotides
  modified_base order(1..3,118..125): mod_base = OTHER; note = Phosphorothioate bond

SEQ ID NO: 134 - RNA, 125 nt, synthetic construct; identical to SEQ ID NO: 34
  source 1..125: mol_type = other RNA
  modified_base order(1..2,119..125): mod_base = OTHER; note = 2-Prime-O-methyl ribonucleotides
  modified_base order(1..3,118..125): mod_base = OTHER; note = Phosphorothioate bond

SEQ ID NO: 135 - RNA, 125 nt, synthetic construct; identical to SEQ ID NO: 38
  source 1..125: mol_type = other RNA
  modified_base order(1..2,119..125): mod_base = OTHER; note = 2-Prime-O-methyl ribonucleotides
  modified_base order(1..3,118..125): mod_base = OTHER; note = Phosphorothioate bond

SEQ ID NO: 136 - RNA, 125 nt, synthetic construct; identical to SEQ ID NO: 42
  source 1..125: mol_type = other RNA
  modified_base order(1..2,119..125): mod_base = OTHER; note = 2-Prime-O-methyl ribonucleotides
  modified_base order(1..3,118..125): mod_base = OTHER; note = Phosphorothioate bond

SEQ ID NO: 137 - RNA, 125 nt, synthetic construct; identical to SEQ ID NO: 46
  source 1..125: mol_type = other RNA
  modified_base order(1..2,119..125): mod_base = OTHER; note = 2-Prime-O-methyl ribonucleotides
  modified_base order(1..3,118..125): mod_base = OTHER; note = Phosphorothioate bond

SEQ ID NO: 138 - DNA, 125 bp, synthetic construct
  source 1..125: mol_type = other DNA
  modified_base 1..3: mod_base = OTHER; note = 2-Prime-O-methyl ribonucleotides
  modified_base 1..4: mod_base = OTHER; note = Phosphorothioate bond
  misc_feature 1..124: note = RNA
  misc_feature 125: note = DNA
tacagctttatgtgactaatgttttagagctagaaatagcaagttaaaataaggctagtccgttatcaacttgaaaaagtggcaccgagtcggtgcggttcccattgaattcagtcacataaagc

SEQ ID NO: 139 - DNA, 125 bp, synthetic construct; identical to SEQ ID NO: 138
  misc_feature 1..123: note = RNA
  source 1..125: mol_type = other DNA
  misc_feature 124..125: note = DNA
  modified_base 1..3: mod_base = OTHER; note = 2-Prime-O-methyl ribonucleotides
  modified_base 1..4: mod_base = OTHER; note = Phosphorothioate bond

SEQ ID NO: 140 - DNA, 125 bp, synthetic construct; identical to SEQ ID NO: 138
  misc_feature 1..122: note = RNA
  source 1..125: mol_type = other DNA
  modified_base 123..125: mod_base = OTHER; note = DNA
  modified_base 1..3: mod_base = OTHER; note = 2-Prime-O-methyl ribonucleotides
  modified_base 1..4: mod_base = OTHER; note = Phosphorothioate bond

SEQ ID NO: 141 - DNA, 125 bp, synthetic construct; identical to SEQ ID NO: 138
  misc_feature 122..125: note = DNA
  source 1..125: mol_type = other DNA
  misc_feature 1..121: note = RNA
  modified_base 1..3: mod_base = OTHER; note = 2-Prime-O-methyl ribonucleotides
  modified_base 1..4: mod_base = OTHER; note = Phosphorothioate bond

SEQ ID NO: 142 - DNA, 125 bp, synthetic construct; identical to SEQ ID NO: 138
  misc_feature 121..125: note = DNA
  source 1..125: mol_type = other DNA
  misc_feature 1..120: note = RNA
  modified_base 1..4: mod_base = OTHER; note = Phosphorothioate bond
  modified_base 1..3: mod_base = OTHER; note = 2-Prime-O-methyl ribonucleotides

SEQ ID NO: 143 - DNA, 125 bp, synthetic construct; identical to SEQ ID NO: 138
  misc_feature 1..119: note = RNA
  source 1..125: mol_type = other DNA
  modified_base 1..4: mod_base = OTHER; note = Phosphorothioate bond
  modified_base 1..3: mod_base = OTHER; note = 2-Prime-O-methyl ribonucleotides
  misc_feature 120..125: note = DNA

SEQ ID NO: 144 - RNA, 125 nt, synthetic construct; identical to SEQ ID NO: 46
  source 1..125: mol_type = other RNA
  modified_base order(1..2,118..125): mod_base = OTHER; note = 2-Prime-O-methyl ribonucleotides
  modified_base order(1..3,118..125): mod_base = OTHER; note = Phosphorothioate bond

SEQ ID NO: 145 - RNA, 125 nt, synthetic construct; identical to SEQ ID NO: 46
  source 1..125: mol_type = other RNA
  modified_base order(1..2,118..125): mod_base = OTHER; note = 2-Prime-O-methyl ribonucleotides
  modified_base order(1..3,119..125): mod_base = OTHER; note = Phosphorothioate bond

SEQ ID NO: 146 - RNA, 125 nt, synthetic construct; identical to SEQ ID NO: 46
  source 1..125: mol_type = other RNA
  modified_base order(1..2,118..125): mod_base = OTHER; note = 2-Prime-O-methyl ribonucleotides
  modified_base order(1..3,120..125): mod_base = OTHER; note = Phosphorothioate bond

SEQ ID NO: 147 - RNA, 125 nt, synthetic construct; identical to SEQ ID NO: 46
  source 1..125: mol_type = other RNA
  modified_base order(1..2,118..125): mod_base = OTHER; note = 2-Prime-O-methyl ribonucleotides
  modified_base order(1..3,121..125): mod_base = OTHER; note = Phosphorothioate bond

SEQ ID NO: 148 - RNA, 125 nt, synthetic construct; identical to SEQ ID NO: 46
  source 1..125: mol_type = other RNA
  modified_base order(1..2,118..125): mod_base = OTHER; note = 2-Prime-O-methyl ribonucleotides
  modified_base order(1..3,122..125): mod_base = OTHER; note = Phosphorothioate bond

SEQ ID NO: 149 - RNA, 125 nt, synthetic construct; identical to SEQ ID NO: 46
  source 1..125: mol_type = other RNA
  modified_base order(1..2,118..125): mod_base = OTHER; note = 2-Prime-O-methyl ribonucleotides
  modified_base order(1..3,123..125): mod_base = OTHER; note = Phosphorothioate bond

SEQ ID NO: 150 - RNA, 125 nt, synthetic construct; identical to SEQ ID NO: 46
  source 1..125: mol_type = other RNA
  modified_base order(1..2,118..125): mod_base = OTHER; note = 2-Prime-O-methyl ribonucleotides
  modified_base order(1..3,124..125): mod_base = OTHER; note = Phosphorothioate bond

SEQ ID NO: 151 - RNA, 125 nt, synthetic construct; identical to SEQ ID NO: 46
  source 1..125: mol_type = other RNA
  modified_base order(1..3,122..124): mod_base = OTHER; note = 2-Prime-O-methyl ribonucleotides
  modified_base order(1..4,122..125): mod_base = OTHER; note = Phosphorothioate bond

(19) United States
(12) Patent Application Publication
(10) Pub. No.: US 2026/0167965 A1
(43) Pub. Date: Jun. 18, 2026

(54) Modified PEG-RNAs with Improved Activity in Prime Editing Applications

(71) Applicant: Integrated DNA Technologies, Inc., Coralville, IA (US)

(72) Inventors: Christopher Anthony VAKULSKAS, North Liberty, IA (US); Michael Allen COLLINGWOOD, Solon, IA (US); Adam BRAINARD, Cedar Rapids, IA (US)

(21) Appl. No.: 19/420,817

(22) Filed: Dec. 16, 2025

Related U.S. Application Data
(60) Provisional application No. 63/735,440, filed on Dec. 18, 2024.

(51) Int. Cl. C12N 15/113; C12N 9/22; C12N 15/86; C12N 15/88; C40B 40/06

(52) U.S. Cl. CPC C12N 15/113; C12N 9/226; C12N 15/86; C12N 15/88; C40B 40/06; C12N 2310/20; C12N 2310/315; C12N 2310/321

(57) ABSTRACT

A modified pegRNA Cas9 nickase system that maintains potent on-target editing activity but has reduced off-target editing activity relative to wild-type Cas, and methods of production and use.

This application claims the benefit of U.S. Ser. No. 63/735,440, filed Dec. 18, 2024, the entirety of which is incorporated herein by reference.

### SPECIFICATION REFERENCE TO AN ELECTRONIC SEQUENCE LISTING

The contents of the electronic sequence listing, 6391-0025PV01.xml; Size: 326,187 bytes; and Date of Creation: Dec. 1, 2024, is herein incorporated by reference in its entirety.

### BACKGROUND

This disclosure pertains to the ability of a nickase CRISPR/Cas9 mutant to cleave double-stranded DNA on one strand in a targeted manner in living cells when complexed with modified prime editing guide RNAs. SpCas9 is an RNA guided endonuclease from the Clustered Regularly Interspaced Short Palindromic Repeat (CRISPR)-Cas (CRISPR-associated) bacterial adaptive immune system of Streptococcus pyogenes (Jinek, M., et al., A programmable dual-RNA-guided DNA endonuclease in adaptive bacterial immunity. Science, 2012. 337 (6096): p. 816-21). Cas9 is guided to a 23-nt DNA target sequence by a target site-specific 20-nt complementary RNA (part of the 44-nt crRNA) and a universal 89-nt tracrRNA, collectively referred to as the guide RNA (gRNA) complex. The Cas9-gRNA ribonucleoprotein (RNP) complex mediates double-stranded DNA breaks (DSBs) which are then typically repaired by the non-homologous end joining (NHEJ), microhomology mediated end joining, or homology-directed repair (HDR) system if a suitable template nucleic acid is present.

S. pyogenes Cas9 protein contains two endonuclease domains that function together to generate a double-strand DNA break by cleaving both the target (guide complementary) and non-target (guide noncomplementary) strands of a double-stranded DNA (dsDNA). These conserved domains are the RuvC and HNH domains. There are two known mutations that can alter Cas9, which natively produces double-stranded cleavage, into a ‘Nickase’ which results in single-stranded cleavage. Cas9 D10A variant generates the nick on the targeted strand, while the Cas9 H840A variant generates the nick on the non-targeted strand (Jinek, M., et al., A programmable dual-RNA-guided DNA endonuclease in adaptive bacterial immunity. Science, 2012. 337 (6096): p. 816-21). The nickase Cas9 variants have been used to facilitate CRISPR-targeted genome editing approaches that do not rely on the introduction of a dsDNA break, examples of which include cytosine/adenine base editors (Komor, A. C., et al., Programmable editing of a target base in genomic DNA without double-stranded DNA cleavage. Nature, 2016. 533 (7603): p. 420-4 and Gaudelli, N. M., et al., Programmable base editing of A*T to G*C in genomic DNA without DNA cleavage. Nature, 2017. 551 (7681): p. 464-471) and more recently the Cas9 prime editor (Anzalone, A. V., et al., Search-and-replace genome editing without double-strand breaks or donor DNA. Nature, 2019. 576 (7785): p. 149-157).

Prime editing is a technology that utilizes a Cas9 nickase fused to an engineered reverse transcriptase. The fusion is coupled with a prime editing guide RNA (pegRNA) that recognizes the target site and contains the desired edit. The prime editor was developed by David Liu and it contains the Cas9 nickase, H840A, and a highly mutagenized reverse transcriptase from Moloney Murine Leukemia Virus (MMLV RTase).

It is understood in the field that CRISPR-Cas9 gRNAs require chemical modification to remain stable for potent genome editing when using DNA, mRNA, viral, and ribonucleoprotein (RNP) delivery modalities. Hendel et al. published a modification pattern that has been accepted as a standard in the field where the 5′ and 3′ termini of the fused single-guide RNA (sgRNA) are modified with phosphorothioate (PS) bonds and 2′o-methylated bases. An example 5′ sgRNA terminus of this standard modification pattern is mG*mC*mU* (first three nucleotides only) and an example of the 3′ terminus is mU*mU*mU*rU (last four nucleotides only). These chemically modified sgRNAs benefit all delivery modalities, which can be important when using expressed delivery formats such as mRNA and DNA (Hendel, A., et al., Chemically modified guide RNAs enhance CRISPR-Cas genome editing in human primary cells. Nat Biotechnol, 2015. 33 (9): p. 985-989).

Although Hendel et al. represents the standard modification pattern and its application to any sgRNA-based technology, the inventors of the instant disclosure have found this modification pattern to be inadequate for potent prime editing activity in living cells. it was further found that the modification pattern that is the most efficacious is not obvious or predictable given prior knowledge of sgRNA modification.

The modified pegRNA Cas9 nickase system as disclosed herein is advantageous for performing prime editing, for example, in a nuclease laden environment such as the inside of a living cell. The modified pegRNA Cas9 nickase system as disclosed herein is advantageous when used for any application that involves prime editing. The patterns of the modified pegRNA systems as disclosed herein can be useful for future Cas9 fusion technologies that rely on an extended sgRNA.

All references cited herein are incorporated herein by reference in their entireties.

### BRIEF SUMMARY

The disclosure provides a modified prime editing guide RNA (“mpegRNA”) nucleotide sequence comprising: i. one or more modified nucleotides at the 5′ end; ii. one or more modified nucleotides at the 3′ end; or iii. one or more modified nucleotides at both the 5′ and 3′ ends, wherein the modified nucleotides comprise a modification selected from the group consisting of 2′-O-methyl modification, phosphorothioate linkage modification, and combinations thereof. The disclosure provides an mpegRNA nucleotide sequence wherein at least two, three, four, five, six, seven, eight, nine, or ten of the nucleotides at the 5′ end of the mpegRNA are modified nucleotides. The disclosure provides an mpegRNA nucleotide sequence comprising modified nucleotides at the 5′ end, wherein the modifications are 3 nucleotides with 2′-O-methyl modification, and 1 nucleotide with a phosphorothioate linkage modification. The disclosure provides an mpegRNA nucleotide sequence comprising modified nucleotides at the 5′ end, wherein the modifications are 2 nucleotides with 2′-O-methyl modification, and 2 nucleotides with a phosphorothioate linkage modification. The disclosure provides an mpegRNA nucleotide sequence wherein at least two, three, four, five, six, seven, eight, nine, or ten of the nucleotides at the 3′ end are modified nucleotides. The disclosure provides an mpegRNA nucleotide sequence comprising modified nucleotides at the 3′ end, wherein the modifications are 7 nucleotides with 2′-O-methyl modifications and 7 nucleotides with phosphorothioate linkage modifications. The disclosure provides an mpegRNA nucleotide sequence comprising modified nucleotides at the 3′ end, wherein the modifications are 6 nucleotides with 2′-O-methyl modifications and 1 nucleotide with phosphorothioate linkage modifications. The disclosure provides an mpegRNA nucleotide sequence comprising modified nucleotides at the 3′ end, wherein the modifications are 5 nucleotides with 2′-O-methyl modifications and 1 nucleotide with phosphorothioate linkage modifications. The disclosure provides an mpegRNA nucleotide sequence comprising selected from the group consisting of: i) a nucleotide sequence comprising modified nucleotides at the 5′ end, wherein the modifications are 3 nucleotides with 2′-O-methyl modification, and 1 nucleotide with a phosphorothioate linkage modification; ii) a nucleotide sequence comprising modified nucleotides at the 5′ end, wherein the modifications are 2 nucleotides with 2′-O-methyl modification, and 2 nucleotides with a phosphorothioate linkage modification; iii) a nucleotide sequence comprising modified nucleotides at the 3′ end, wherein the modifications are 7 nucleotides with 2′-O-methyl modifications and 7 nucleotides with phosphorothioate linkage modifications; iv) a nucleotide sequence comprising modified nucleotides at the 3′ end, wherein the modifications are 6 nucleotides with 2′-O-methyl modifications and 1 nucleotide with phosphorothioate linkage modifications; v) a nucleotide sequence comprising modified nucleotides at the 3′ end, wherein the modifications are 5 nucleotides with 2′-O-methyl modifications and 1 nucleotide with phosphorothioate linkage modifications; and vi) any combinations thereof.

The disclosure provides a modified prime editing guide RNA (“mpegRNA”) comprising: i. a targeting sequence comprising one or more modified nucleotides at the 5′ end; ii. a sgRNA scaffold; iii. a reverse transcriptase template with a desired edit sequence; and iv. a primer binding site comprising one or more modified nucleotides at the 3′ end, wherein the one or more modified nucleotides comprise at least one modification selected from the group consisting of 2′-O-methyl modification, phosphorothioate linkage modification, and combinations thereof. In certain embodiments the disclosure provides a modified prime editing guide RNA (“mpegRNA”) comprising: i) a targeting sequence comprising one or more modified nucleotides at the 5′ end; and ii) a primer binding site comprising one or more modified nucleotides at the 3′ end, wherein the one or more modified nucleotides comprise at least one modification selected from the group consisting of 2′-O-methyl modification, phosphorothioate linkage modification, and combinations thereof. The disclosure provides an mpegRNA nucleotide sequence wherein the targeting sequence comprises at least two, three, four, five, six, seven, eight, nine, or ten modified nucleotides. The disclosure provides an mpegRNA nucleotide sequence wherein the targeting sequence comprises three nucleotides with 2′-O-methyl modification, and one nucleotide with a phosphorothioate linkage modification. The disclosure provides an mpegRNA nucleotide sequence wherein the targeting sequence comprises two nucleotides with 2′-O-methyl modification, and two nucleotides with a phosphorothioate linkage modification. The disclosure provides an mpegRNA nucleotide sequence wherein the primer binding site comprises at least two, three, four, five, six, seven, eight, nine, or ten modified nucleotides. The disclosure provides an mpegRNA nucleotide sequence wherein the primer binding site comprises seven nucleotides with 2′-O-methyl modifications and seven nucleotides with phosphorothioate linkage modifications. The disclosure provides an mpegRNA nucleotide sequence wherein the primer binding site comprises six nucleotides with 2′-O-methyl modifications and one nucleotide with phosphorothioate linkage modifications. The disclosure provides an mpegRNA nucleotide sequence wherein the primer binding site comprises five nucleotides with 2′-O-methyl modifications and one nucleotide with phosphorothioate linkage modifications. The disclosure provides an mpegRNA nucleotide sequence comprising selected from the group consisting of: vii) a targeting sequence comprising three nucleotides with 2′-O-methyl modifications and one nucleotide with a phosphorothioate linkage modification; viii) a targeting sequence comprising two nucleotides with 2′-O-methyl modifications and two nucleotides with a phosphorothioate linkage modifications; ix) a primer binding site comprising seven nucleotides with 2′-O-methyl modifications and seven nucleotides with phosphorothioate linkage modifications; x) a primer binding site comprising six nucleotides with 2′-O-methyl modifications and one nucleotide with phosphorothioate linkage modifications; xi) a primer binding site comprising five nucleotides with 2′-O-methyl modifications and one nucleotide with phosphorothioate linkage modification; and xii) any combinations thereof. The disclosure provides an mpegRNA nucleotide sequence wherein the mpegRNA comprises five nucleotides with 2′-O-methyl modifications and one nucleotide with phosphorothioate linkage modifications at the 3′ end, and wherein the mpegRNA comprises two nucleotides with 2′-O-methyl modifications and two nucleotides with phosphorothioate linkage modifications at the 5′ end. The disclosure provides an mpegRNA nucleotide sequence wherein the mpegRNA comprises six nucleotides with 2′-O-methyl modifications and one nucleotide with phosphorothioate linkage modifications at the 3′ end, and wherein the mpegRNA comprises two nucleotides with 2′-O-methyl modifications and two nucleotides with phosphorothioate linkage modifications at the 5′ end. The disclosure provides an mpegRNA nucleotide sequence wherein off-target editing relative to an unmodified gRNA is reduced by an amount selected from the group consisting of 80%, 85%, 90%, 95%, and 99%. The disclosure provides an mpegRNA nucleotide sequence wherein the at least one modified nucleotide alters base-pairing thermostability. The disclosure provides an mpegRNA nucleotide sequence wherein said at least one modified nucleotide enhances base-pairing thermostability. The disclosure provides an mpegRNA nucleotide sequence wherein said at least one modified nucleotide decreases base-pairing thermostability. The disclosure provides an mpegRNA nucleotide sequence wherein the at least one modified nucleotide is a specificity-altering modification. The disclosure provides an mpegRNA nucleotide sequence wherein the modified mpegRNA is chemically synthesized.

The disclosure provides a set or library of RNA molecules comprising two or more mpegRNAs as disclosed herein. The disclosure provides a kit comprising the mpegRNA as disclosed herein. The disclosure provides an array of RNA molecules comprising two or more mpegRNAs as disclosed herein.

The disclosure provides a method for inducing gene regulation of a target nucleic acid in a cell, the method comprising: introducing into the cell: (a) an mpegRNA as disclosed herein; (b) a Cas9 polypeptide, an mRNA encoding a Cas9 polypeptide, or a recombinant expression vector comprising a nucleotide sequence encoding a Cas9 polypeptide, wherein the mpegRNA guides the Cas9 polypeptide to the target nucleic acid, and wherein the mpegRNA induces a gene regulation of the target nucleic acid with an enhanced activity relative to a corresponding unmodified mpegRNA. The disclosure provides a method for inducing gene regulation of a target nucleic acid in a cell wherein the modified mpegRNA in (a) and the Cas9 polypeptide in (b) are introduced into the cell in a ribonucleoprotein (RNP) complex. The disclosure provides a method for inducing gene regulation of a target nucleic acid in a cell wherein the gene regulation induced by the introduction of (a) and (b) is stable in the cell for at least 24 hours. The disclosure provides a method for inducing gene regulation of a target nucleic acid in a cell wherein the mpegRNA in (a) and the Cas9 polypeptide in (b) are introduced into the cell in a lipofection reagent. The disclosure provides a method for inducing gene regulation of a target nucleic acid in a cell wherein the mpegRNA in (a) and the Cas9 polypeptide in (b) are introduced into the cell via exosomes. The disclosure provides a method for inducing gene regulation of a target nucleic acid in a cell wherein the mpegRNA in (a) and the Cas9 polypeptide in (b) are introduced into the cell via lipid nanoparticles. The disclosure provides a method for inducing gene regulation of a target nucleic acid in a cell wherein the mpegRNA in (a) and the Cas9 polypeptide in (b) are introduced into the cell via viral vector.

### BRIEF DESCRIPTION OF SEVERAL VIEWS OF THE DRAWINGS

The invention will be described in conjunction with the following drawings in which like reference numerals designate like elements and wherein:

FIG. 1 is a chart showing that representative pegRNA modification patterns show greatly improved efficacy over the Standard Modification pattern as disclosed in Hendel et al (“Positive Control”). Positive Control is represented by SEQ ID No: 151, M24 is represented by SEQ ID No: 73, and M115 is represented by SEQ ID No: 137. Negative Control=cell only.

FIG. 2 is a graphic illustration of M115 showing representative regions within M115 (chemical modifications have been omitted).

### DETAILED DESCRIPTION

The Prime Editor nuclease fusion utilizes a Cas9 sgRNA fused to a primer binding site (PBS) and RTase Template (RTT) which is collectively referred to as a Prime Editing gRNA (PEG-RNA) [4]. Little is understood about chemical modification patterns of pegRNAs that are suitable for the various delivery modalities and given the overall intrinsic low potency of PE systems, pegRNA modification and maintaining RNA stability in vivo are important to this technique's success. Given that the 3′ terminus of PEG RNAs encode an RNA template for priming by the nascent CRISPR break site, it is presently unclear and not obvious what modifications (if any) would be beneficial to PE activity. The disclosure provides modifications that benefit pegRNAs and to find the correct balance between sufficient modification to maintain stability without negatively impacting function of the pegRNA. We find that a certain specific arrangement of chemical modifications that is distinctly different from the standard modification pattern as disclosed in Hendel et al. is both not obvious, and greatly beneficial to potency of this system.

The Cas9 nickase-modified pegRNA system as disclosed herein can be used for any application that involves prime editing in a nuclease laden environment such as the inside of a living cell. The patterns of the modified pegRNA systems as disclosed herein can be useful for future Cas9 fusion technologies that rely on an extended sgRNA.

The Clustered Regularly Interspaced Short Palindromic Repeats and CRISPR-associated protein 9 (CRISPR-Cas9) system is a tool in genetic engineering to precisely edit genes within organisms. CRISPR-Cas systems are native to bacteria and Archaea to provide adaptive immunity against viruses and plasmids. CRISPR are sequences of DNA in bacterial genomes that store fragments of viral DNA. If the bacterium survives a viral attack, it incorporates a piece of the virus's genetic material into its own DNA. These stored sequences help the bacteria recognize and destroy a virus or plasmid. Cas9 is an enzyme that cuts DNA at specific sites. It's guided by a piece of RNA, known as guide RNA (gRNA), which matches a specific DNA sequence that scientists want to modify. The gRNA is complementary to the DNA sequence to target. This gRNA will guide the Cas9 protein to the exact location in the genome. Once guided to the target location, the Cas9 enzyme cuts the DNA at that precise spot. After Cas9 cuts the DNA, the cell tries to repair the break. Providing a template with the desired DNA sequence can instruct the cell to incorporate this new sequence during the repair process.

There are three classes of CRISPR-Cas systems that could potentially be adapted for research and therapeutic reagents, but Type-II CRISPR systems have a desirable characteristic in utilizing a single CRISPR associated (Cas) nuclease (specifically Cas9) in a complex with the appropriate guide RNAs—either a 2-part RNA system similar to the natural complex in bacteria comprising a CRISPR-activating RNA:trans-activating crRNA (crRNA:tracrRNA) pair or an artificial chimeric single-guide-RNA (sgRNA)—to mediate double-stranded cleavage of target DNA. In mammalian systems, these RNAs have been introduced by transfection of DNA cassettes containing RNA Pol III promoters (such as U6 or H1) driving RNA transcription, viral vectors, and single-stranded RNA following in vitro transcription (see Xu, T., et al., Appl Environ Microbiol, 2014. 80 (5): p. 1544-52).

Cas9 nickase is a modified version of the Cas9 protein. While the wild-type Cas9 enzyme introduces double-strand breaks (DSBs) in DNA, Cas9 nickase is designed to make a single-strand break, or “nick,” at a specific location on the DNA. This modified version offers more precision and reduces the risk of unintended genomic alterations (off-target effects). Cas9 nickase only cuts one strand of the DNA, creating a nick rather than a complete break. This happens by mutating one of Cas9's two nuclease domains, either RuvC or HNH, which renders it inactive. As a result, Cas9 retains the ability to cut only one DNA strand. For genome editing, two Cas9 nickases can be used in combination to target opposite strands of DNA close to each other. This creates two single-strand breaks, which can then be treated as a DSB by the cell's repair machinery, which may reduce the likelihood of off-target effects. DSBs are typically repaired by non-homologous end joining (NHEJ), which is error-prone and can lead to insertions or deletions (indels). Nicks, on the other hand, are primarily repaired through homology-directed repair (HDR), a more accurate repair process if a homologous DNA template is available. The nickase system allows for more precise editing with fewer chances of errors or unwanted mutations, especially when using HDR for precise gene modifications.

Cas9 has two nuclease domains, RuvC and HNH, that are responsible for cleaving the two strands of DNA. To create a nickase, one of these domains is mutated: Mutating the RuvC domain (D10A mutation) leads to the nicking of the target (guide complementary) strand only. Mutating the HNH domain (H840A mutation) results in the nicking of the non-target (guide noncomplementary) strand only.

Paired nickases can be used to introduce highly accurate modifications by ensuring both strands are cut only at the intended target sites. Since two nicks are required to create a DSB, the probability of off-target double-strand breaks is significantly reduced compared to wild-type Cas9. Cas9 nickase can be combined with deaminases for base editing, allowing the precise conversion of one base to another (e.g., C to T) without inducing a DSB.

Prime editing is an advanced genome editing technique derived from CRISPR technology which offers the ability to precisely insert, delete, or alter specific DNA sequences without the need for double-strand breaks (DSBs) or donor DNA templates. The Components of Prime Editing include a Cas9 Nickase (Cas9n). Other components include Reverse Transcriptase (RT). In prime editing, the Cas9 nickase is fused to a reverse transcriptase enzyme, which synthesizes a new DNA strand using an RNA template provided by the pegRNA (prime editing guide RNA). This allows for precise edits, including base changes, insertions, or deletions, which may be near and/or directly at the nicked site. The Prime Editing Guide RNA (pegRNA) is an extended version of the traditional guide RNA (gRNA) which has two main parts: A guide sequence that directs the Cas9 nickase to the correct location in the genome, ensuring the cut happens at the desired site; and a reverse transcriptase template and a primer binding site that encode the desired DNA sequence changes. This template provides the instructions for the reverse transcriptase to make the new DNA sequence, which will replace the original DNA at the target site.

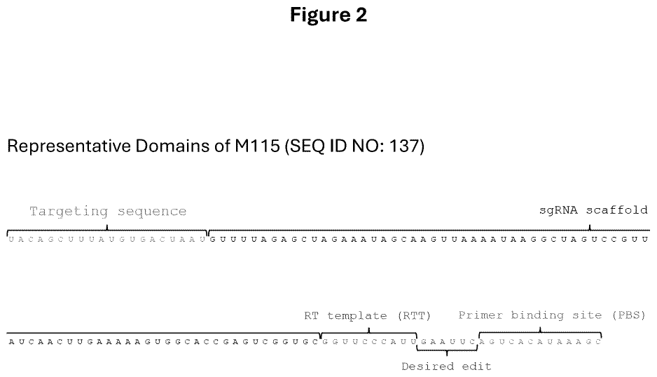

The pegRNA guides the Cas9 nickase to a specific location in the genome, where it makes a single-strand nick in the DNA. After the DNA is nicked, the reverse transcriptase enzyme uses the reverse transcription template in the pegRNA to synthesize a complementary DNA sequence. This new DNA strand incorporates the desired genetic change (such as a base substitution, insertion, or deletion). Prime editing allows for very precise genome modifications, including base substitutions, small insertions, deletions, and even complex modifications like transversions (e.g., changing a C-G pair to a G-C pair). A pegRNA includes a targeting sequence which is generally a 20-nucleotide sequence that guides the CRISPR-Cas9 complex to the specific DNA sequence to be edited; a sgRNA scaffold sequence which binds to the Cas9 nickase. The Cas9 nickase remains inactive on the opposite strand, allowing precise single-strand cutting rather than double-strand breaks; a reverse transcriptase template (RTT) which contains the new, desired DNA sequence to be inserted, deleted, or substituted in the genome; and a primer binding site (PBS) which is located at the 3′ end, this region allows the pegRNA to bind to the single-stranded DNA cut by Cas9 nickase. The PBS is essential for initiating the synthesis of new DNA with the desired edit (see e.g., FIG. 2).

In some embodiments, RNA modifications that can be used in the modified prime editing guide RNA (“mpegRNA”) system and methods of the instant disclosure include, for example: Sugar Modifications include, for example, 2′-O-Methyl(2′-OMe); 2′-Fluoro (2′-F); 2′-Amino (2′-NH2); 2′-O-Methoxyethyl(2′-MOE); Locked Nucleic Acid (LNA); Unlocked Nucleic Acids (UNA). Backbone Modifications include, for example, Phosphorothioate (PS) Bonds, which replace a non-bridging oxygen in the phosphate backbone with sulfur, enhancing resistance to nuclease degradation and improving pharmacokinetics; Phosphorodiamidate Morpholino Oligomers (PMO); Methylphosphonate Linkages; Boranophosphate. Base Modifications include, for example, 5-Methylcytidine (5mC); N6-Methyladenosine (m6A); Pseudouridine (Y); 5-Hydroxymethylcytosine (hm5C); Inosine (I); 7-Methylguanosine (m7G). Cap and Tail Modifications include, for example, 5′ Cap Structures (e.g., m7G cap); Poly (A) Tail; Antisense Oligonucleotide (ASO) Tail Modifications. Conjugated Modifications include, for example, Lipophilic Conjugates (e.g., cholesterol, fatty acids); PEGylation (Polyethylene Glycol); Targeting Ligands (e.g., GalNAc, folate). Additional modifications for reducing immunogenicity include, for example, 2′-Deoxy-2′-fluoro-arabinonucleic Acid (FANA); 2′-Deoxyuridine; Base analogs (e.g., 5-bromo, 5-iodo modifications). In certain embodiments as disclosed herein, the mpegRNAs as disclosed herein can be chemically synthesized.

The modified pegRNA system components as disclosed herein, for example, including a Cas9 nickase, can be introduced into the cell, together or separately, using various approaches. Examples include plasmid or viral expression vectors (which lead to endogenous expression), Cas9 mRNA with separate mpegRNA transfection, or delivery of the Cas9 protein with the mpegRNA as a ribonucleoprotein (RNP) complex (see Kouranova et al., Hum Gen Ther (2016) 27 (6): 464-475). Effective strategies for introducing, for example, the Cas9 nickase-mpegRNA system as disclosed herein into target cells include, for example, viral vectors, such as Adeno-Associated Virus (AAV) which are widely used for CRISPR delivery because they are generally safe, induce minimal immune response, and have been approved in some gene therapy applications; lentivirus and retrovirus; and adenovirus.

Additional methods for introducing the modified pegRNA system components as disclosed herein, for example, including a Cas9 nickase, into target cells includes, for example, Lipid Nanoparticles (LNPs) which are commonly used for delivering RNA-based therapies; Electroporation, which involves applying an electrical field to create temporary pores in the cell membrane, allowing CRISPR components (like plasmids, ribonucleoprotein complexes, or mRNA, such as mpegRNA) to enter the cell. Additional methods for introducing the modified pegRNA system components as disclosed herein, for example, including a Cas9 nickase, into target cells includes, for example, Ribonucleoprotein (RNP) Complexes which involves directly delivering the Cas9 protein pre-complexed with mpegRNA into cells, usually via electroporation or lipid-based transfection; Lipid-Based Transfection Agents (lipofection) uses lipid-based reagents to encapsulate CRISPR plasmids or RNP complexes and facilitate their uptake by cells.

Other methods for introducing the modified pegRNA system components as disclosed herein, for example, including a Cas9 nickase, into target cells includes, for example, physical methods such as microinjection to directly inject CRISPR components into cells, typically used in single-cell embryos or zygotes for generating transgenic animals; nanoneedles and microfluidics which can introduce CRISPR components with minimal damage to cells; and exosome-mediated delivery, which can be engineered to carry CRISPR/Cas components and target them to specific cells.

Next Generation Sequencing (NGS) allows rapid and high-throughput sequencing of DNA and RNA. Unlike earlier methods such as Sanger sequencing, which sequences one DNA fragment at a time, NGS enables the simultaneous sequencing of millions of DNA fragments, making it much faster, cheaper, and more efficient. In NGS, a DNA or RNA from the sample is extracted and fragmented into smaller pieces. These fragments are then attached to short synthetic DNA sequences called adapters, which are needed for binding to the sequencing platform. The DNA fragments with adapters are amplified (copied many times) to create a “library” of DNA fragments. This increases the amount of DNA available for sequencing. Most NGS platforms, like Illumina, use a method called “sequencing by synthesis.” Each fragment is attached to a solid surface and copied in place. Fluorescently labeled nucleotides (A, T, C, and G) are added one by one. As they bind to the complementary strand, the machine detects the fluorescent signal, allowing the sequence of bases to be read. The massive amount of sequencing data is analyzed using bioinformatics tools. The overlapping DNA fragments are assembled back into their original sequence by aligning them to a reference genome or constructing new genomes (de novo sequencing). NGS is high throughput, since millions to billions of DNA fragments can be sequenced in parallel, producing vast amounts of data, is cost-effective, and can sequence entire genomes or large sets of genes in days, making it much faster than older sequencing methods. Tiled-amplicon NGS is a targeted sequencing method designed to analyze specific regions of a genome or set of genes by amplifying overlapping “tiles” of DNA fragments through polymerase chain reaction (PCR). Each tile covers a portion of the target region, and their overlapping design ensures comprehensive coverage and accurate sequence reconstruction. In tiled-amplicon NGS target regions of interest are divided into overlapping amplicon segments and primers are designed to amplify these amplicons, ensuring overlap between adjacent segments to cover the entire target area. The primers amplify their corresponding tiles using PCR, creating a library of DNA fragments from the target regions. Multiplex PCR is often used, where multiple amplicons are amplified simultaneously in a single reaction. The amplified DNA is prepared for sequencing by adding adapters and barcodes for sample identification and compatibility with the sequencing platform. The prepared DNA library is sequenced on an NGS platform, generating large volumes of short-read sequence data. Sequencing reads are aligned to a reference genome or assembled de novo to reconstruct the target regions. Overlapping tiles help resolve sequencing errors, fill gaps, and increase the accuracy of variant detection.

A variety of host cells can serve as platforms for CRISPR expression, such as: Bacterial Cells: Escherichia coli is widely used for CRISPR applications like plasmid construction, cloning, and CRISPR screens; Yeast: Saccharomyces cerevisiae and other yeast species can be genetically modified to express CRISPR systems, especially in studies focused on gene function and genome screening in eukaryotic systems; Insect cells can serve as hosts for CRISPR expression. In particular, insect cell lines such as Sf9 (from Spodoptera frugiperda) and S2 (from Drosophila melanogaster); Mammalian Cells: Various mammalian cell lines, including HEK293, HeLa, and CHO cells, are commonly used, which are compatible with more complex CRISPR modifications, such as large gene insertions, knock-ins, or base editing, due to their complex regulatory machinery; Primary Cells and Stem Cells: Primary cells, like human or animal-derived cells, and induced pluripotent stem cells (iPSCs) can also be used as host cells for CRISPR systems, especially for therapeutic studies and disease modeling; Plant Cells: Plants like Arabidopsis thaliana, tobacco, and rice can serve as CRISPR host cells. Plant cells are often transformed with CRISPR machinery to study gene function, enhance traits, or improve resistance to pathogens. Various delivery systems for CRISPR components, such as plasmids, viral vectors, or ribonucleoprotein complexes, and specific promoters can be optimized for the host's transcriptional machinery.

The term “nucleic acid” refers to a nucleotide polymer, and unless otherwise limited, includes analogs of natural nucleotides that can function in a similar manner (e.g., hybridize) to naturally occurring nucleotides. The term “nucleic acid” encompasses multi-stranded, as well as single-stranded molecules. In double- or triple-stranded nucleic acids, the nucleic acid strands need not be coextensive (i.e., a double-stranded nucleic acid need not be double-stranded along the entire length of both strands). Nucleic acid templates described herein may be any size depending on the sample (from small cell-free DNA fragments to entire genomes), including but not limited to 50-300 bases, 100-2000 bases, 100-750 bases, 170-500 bases, 100-5000 bases, 50-10,000 bases, or 50-2000 bases in length. In some instances, templates are at least 50, 100, 200, 500, 1000, 2000, 5000, 10,000, 20,000 50,000, 100,000, 200,000, 500,000, 1,000,000 or more than 1,000,000 bases in length. Methods described herein provide for the amplification of nucleic acids, such as nucleic acid templates. Methods described herein additionally provide for the generation of isolated and at least partially purified nucleic acids and libraries of nucleic acids. Nucleic acids include but are not limited to those comprising DNA, RNA, circular RNA, cfDNA (cell free DNA), cfRNA (cell free RNA), siRNA (small interfering RNA), cffDNA (cell free fetal DNA), mRNA, tRNA, IRNA, miRNA (microRNA), synthetic polynucleotides, polynucleotide analogues, any other nucleic acid consistent with the specification, or any combinations thereof. The length of polynucleotides, when provided, are described as the number of bases and abbreviated, such as nt (nucleotides), bp (bases), kb (kilobases), or Gb (gigabases).

The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; mRNA; and non-coding RNA.

The term nucleic acid encompasses double- or triple-stranded nucleic acid complexes, as well as single-stranded molecules. In double- or triple-stranded nucleic acid complexes, the nucleic acid strands need not be coextensive (i.e, a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like. More particularly, in some embodiments, nucleic acids, can include polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholino Antisense Oligomers: Design, Preparation, and Properties,” Antisense & Nucleic Acid Drug Dev. 7:1817-195; Okamoto et al. (20020) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses, for example, locked nucleic acids (LNAs) and unlocked nucleic acids (UNAs).

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “complementary” refers to the capacity for precise pairing between two nucleotides, i.e., if a nucleotide at a given position of a nucleic acid is capable of hydrogen bonding with a nucleotide of another nucleic acid to form a canonical base pair, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules. The term “oligonucleotide,” as used herein, refer to polydeoxyribonucleotides (containing 2-deoxy-D-ribose), polyribonucleotides (containing D-ribose), and to any other type of polynucleotide which is an N glycoside of a purine or pyrimidine base (a single nucleotide is also referred to as a “base” or “residue”). There is no intended distinction in length between the terms “nucleic acid”, “oligonucleotide” and “polynucleotide”, and these terms can be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar, or phosphate backbone is modified as well as non-purine or non-pyrimidine nucleotide analogs. An oligonucleotide may comprise ribonucleotides, deoxyribonucleotides, modified nucleotides (e.g., nucleotides with 2′ modifications, synthetic base analogs, etc.) or combinations thereof.

The term “ribonucleotide” encompasses natural and synthetic, unmodified and modified ribonucleotides. Modifications include changes to the sugar moiety, to the base moiety, and/or to the linkages between ribonucleotides in the oligonucleotide.

The term “polypeptide” refers to any linear or branched peptide comprising more than one amino acid. Polypeptide includes protein or fragment thereof or fusion thereof, provided such protein, fragment or fusion retains a useful biochemical or biological activity.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example, “1, 2 and/or 3” is equivalent to “1, 2, 3, 1 and 2, 1 and 3, 2 and 3, or 1, 2, and 3”.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The disclosure as illustratively described herein suitably may be practiced in the absence of any element, elements, limitation, or limitations which is not specifically disclosed herein.

As used herein and in the appended claims, the singular forms “a”, “an”, and “the” include plural referents unless the context clearly dictates otherwise. Thus, for example, reference to “a method” includes a plurality of such methods and reference to “the nanoparticle” includes reference to one or more nanoparticles and equivalents thereof known to those skilled in the art, and so forth. The terms and expressions which have been employed are used as terms of description and not of limitation, and there is no intention in the use of such terms and expressions of excluding any equivalents of the features shown and described or portions thereof, but it is recognized that various modifications are possible within the scope as disclosed herein claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All references throughout this application, for example patent documents, including issued or granted patents or equivalents and patent application publications, and non-patent literature documents or other source material are hereby incorporated by reference herein in their entireties, as though individually incorporated by reference. None is admitted to be prior art.

As used herein, the term “about” when used in conjunction with a stated numerical value or range has the meaning reasonably ascribed to it by a person skilled in the art, i.e., denoting somewhat more or somewhat less than the stated value or range.

The invention will be illustrated in more detail with reference to the following Examples, but it should be understood that the present invention is not deemed to be limited thereto.

### EXAMPLES

### Example 1—Specific Arrangements of PS and 2′-O-Methyl Bases to the Terminus of PEG RNAs Greatly Improves Efficacy

We initially targeted the HPRT-38673 locus with a PEG RNA that inserts an EcoRI site (GAATTC) and tested increasing PS bonds and 2′-O-methylation at the 5′ and 3′ termini. The PEG RNAs used in this study were chemically synthesized by Integrated DNA Technologies as custom guide RNA products (Table 1). Plasmid expressing prime editor protein and chemically synthesized mpegRNAs were introduced into HEK293 cells by electroporation (Lonza Nucleofector) and genomic DNA was extracted after 72 hr. Insertion of the EcoRI sequence was assessed by restriction digest and DNA analysis on the Fragment Analyzer (Agilent). The best performing modification patterns at HPRT-38673 were extrapolated to 11 additional loci at HPRT and were shown to be universally beneficial at these sites where detectable editing was present.

TABLE 1
Sequences of PEG-RNAs tested

| Seq_ID | Sequence |
|---|---|
| SEQ ID NO: 1 | mG*mG*mC*rCrCrArGrArCrUrGrArGrCrArCrGrUrGrArGrUrUrUrUrArGrArGrCrUrArGrAr |
| | ArArUrArGrCrArArGrUrUrArArArArUrArArGrGrCrUrArGrUrCrCrGrUrUrArUrCrArArCrUrUr |
| | GrArArArArArGrUrGrGrGrArCrCrGrArGrUrCrGrGrUrCrCrUrCrUrGrCrCrArUrCrArGrArArU |
| | rUrCrCrGrUrGrCrUrCrArGrU*mC*mU*mG |
| SEQ ID NO: 2 | mA*mA*mU* rUrArU rGrGrG rGrArU rUrArC rUrArG rGrArG rUrUrU rUrArG rArGrC |
| | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rUrGrC |
| | rCrCrC rUrUrC rCrGrA rArUrU rCrUrA rGrUrA rArUrC rCrC*mC* mA*mU |
| SEQ ID NO: 3 | mA*mA*mU* rUrArU rGrGrG rGrArU rUrArC rUrArG rGrArG rUrUrU rUrArG rArGrC |
| | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rUrGrC |
| | rUrGrC rCrCrC rUrUrC rCrGrA rArUrU rCrUrA rGrUrA rArUrC rCrC*mC* mA*mU |
| SEQ ID NO: 4 | mA*mA*mU* rUrArU rGrGrG rGrArU rUrArC rUrArG rGrArG rUrUrU rUrArG rArGrC |
| | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rUrGrC |
| | rCrCrC rUrUrC rCrGrA rArUrU rCrUrA rGrUrA rArUrC rCrC*mC* mA*mU |
| SEQ ID NO: 5 | mA*mA*mU* rUrArU rGrGrG rGrArU rUrArC rUrArG rGrArG rUrUrU rUrArG rArGrC |
| | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rUrGrC |
| | rUrGrC rCrCrC rUrUrC rCrGrA rArUrU rCrUrA rGrUrA rArUrC rCrC*mC* mA*mU |
| SEQ ID NO: 6 | mU*mC*mC* rArUrU rUrCrA rUrArG rUrCrU rUrUrC rCrUrG rUrUrU rUrArG rArGrC |
| | rUrArG rArArA rUrArG rCrArA rGrUrU rArArA rArUrA rArGrG rCrUrA rGrUrC rCrGrUrUrArU |
| | rCrArA rCrUrU rGrArA rArArA rGrUrG rGrCrA rCrCrG rArGrU rCrGrG rUrGrC rArCrA |
| | rCrCrC rArArG rGrGrA rArUrU rCrArA rArGrA rCrUrA rUrG*mA* mA*mA |
| SEQ ID NO: 7 | mU*mC*mC* rArUrU rUrCrA rUrArG rUrCrU rUrUrC rCrUrG rUrUrU rUrArG rArGrC |
| | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rArArC |
| | rArCrA rCrCrC rArArG rGrGrA rArUrU rCrArA rArGrA rCrUrA rUrG*mA* mA*mA |
| SEQ ID NO: 8 | mU*mC*mC* rArUrU rUrCrA rUrArG rUrCrU rUrUrC rCrUrG rUrUrU rUrArG rArGrC |
| | rUrArG rArArA rUrArG rCrArA rGrUrU rArArA rArUrA rArGrG rCrUrA rGrUrC rCrGrUrUrArU |
| | rCrArA rCrUrU rGrArA rArArA rGrUrG rGrGrA rCrCrG rArGrU rCrGrG rUrCrC rArCrA |
| | rCrCrC rArArG rGrGrA rArUrU rCrArA rArGrA rCrUrA rUrG*mA* mA*mA |
| SEQ ID NO: 9 | mU*mC*mC* rArUrU rUrCrA rUrArG rUrCrU rUrUrC rCrUrG rUrUrU rUrArG rArGrC |
| | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rArArC |
| | rArCrA rCrCrC rArArG rGrGrA rArUrU rCrArA rArGrA rCrUrA rUrG*mA* mA*mA |
| SEQ ID NO: | mG*mG*mU* rCrArC rUrUrU rUrArA rCrArC rArCrC rCrArG rUrUrU rUrArG rArGrC |
| 10 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rCrUrU |
| | rUrCrC rUrUrG rGrGrA rArUrU rCrGrU rGrUrG rUrUrA rArA*mA* mG*mU |
| SEQ ID NO: | mG*mG*mU* rCrArC rUrUrU rUrArA rCrArC rArCrC rCrArG rUrUrU rUrArG rArGrC |
| 11 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rArGrU |
| | rCrUrU rUrCrC rUrUrG rGrGrA rArUrU rCrGrU rGrUrG rUrUrA rArA*mA* mG*mU |
| SEQ ID NO: | mG*mG*mU* rCrArC rUrUrU rUrArA rCrArC rArCrC rCrArG rUrUrU rUrArG rArGrC |
| 12 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rCrUrU |
| | rUrCrC rUrUrG rGrGrA rArUrU rCrGrU rGrUrG rUrUrA rArA*mA* mG*mU |
| SEQ ID NO: | mG*mG*mU* rCrArC rUrUrUrUrArA rCrArC rArCrC rCrArG rUrUrU rUrArG rArGrC |
| 13 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rArGrU |
| | rCrUrU rUrCrC rUrUrG rGrGrA rArUrU rCrGrU rGrUrG rUrUrA rArA*mA* mG*mU |
| SEQ ID NO: | mU*mU*mU* rUrGrU rArArU rUrArA rCrArG rCrUrU rGrCrG rUrUrU rUrArG rArGrC |
| 14 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rUrUrC |
| | rArCrC rArGrC rArGrA rArUrU rCrArG rCrUrG rUrUrA rArU*mU* mA*mC |
| SEQ ID NO: | mU*mU*mU* rUrGrU rArArU rUrArA rCrArG rCrUrU rGrCrG rUrUrU rUrArG rArGrC |
| 15 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rCrUrU |
| | rUrUrC rArCrC rArGrC rArGrA rArUrU rCrArG rCrUrG rUrUrA rArU*mU* mA*mC |
| SEQ ID NO: | mU*mU*mU* rUrGrU rArArU rUrArA rCrArG rCrUrU rGrCrG rUrUrU rUrArG rArGrC |
| 16 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rUrUrC |
| | rArCrC rArGrC rArGrA rArUrU rCrArG rCrUrG rUrUrA rArU*mU* mA*mC |
| SEQ ID NO: | mU*mU*mU* rUrGrU rArArU rUrArA rCrArG rCrUrU rGrCrG rUrUrU rUrArG rArGrC |
| 17 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rCrUrU |
| | rUrUrC rArCrC rArGrC rArGrA rArUrU rCrArG rCrUrG rUrUrA rArU*mU* mA*mC |
| SEQ ID NO: | mC*mU*mU* rArUrA rUrCrC rArArC rArCrU rUrCrG rUrGrG rUrUrU rUrArG rArGrC |
| 18 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rArGrG |
| | rArCrC rCrCrA rCrGrA rArUrU rCrGrA rArGrU rGrUrU rGrG*mA* mU*mA |
| SEQ ID NO: | mC*mU*mU* rArUrA rUrCrC rArArC rArCrU rUrCrG rUrGrG rUrUrU rUrArG rArGrC |
| 19 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rArArA |
| | rArGrG rArCrC rCrCrA rCrGrA rArUrU rCrGrA rArGrU rGrUrU rGrG*mA* mU*mA |
| SEQ ID NO: | mC*mU*mU* rArUrA rUrCrC rArArC rArCrU rUrCrG rUrGrG rUrUrU rUrArG rArGrC |
| 20 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rArGrG |
| | rArCrC rCrCrA rCrGrA rArUrU rCrGrA rArGrU rGrUrU rGrG*mA* mU*mA |
| SEQ ID NO: | mC*mU*mU* rArUrA rUrCrC rArArC rArCrU rUrCrG rUrGrG rUrUrU rUrArG rArGrC |
| 21 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rArArA |
| | rArGrG rArCrC rCrCrA rCrGrA rArUrU rCrGrA rArGrU rGrUrU rGrG*mA* mU*mA |
| SEQ ID NO: | mG*mG*mC* rUrUrA rUrArU rCrCrA rArCrA rCrUrU rCrGrG rUrUrU rUrArG rArGrC |
| 22 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrArC |
| | rCrCrC rArCrG rArGrA rArUrU rCrArG rUrGrU rUrGrG rArU*mA* mU*mA |
| SEQ ID NO: | mG*mG*mC* rUrUrA rUrArU rCrCrA rArCrA rCrUrU rCrGrG rUrUrU rUrArG rArGrC |
| 23 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rArArG |
| | rGrArC rCrCrC rArCrG rArGrA rArUrU rCrArG rUrGrU rUrGrG rArU*mA* mU*mA |
| SEQ ID NO: | mG*mG*mC* rUrUrA rUrArU rCrCrA rArCrA rCrUrU rCrGrG rUrUrU rUrArG rArGrC |
| 24 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rGrArC |
| | rCrCrC rArCrG rArGrA rArUrU rCrArG rUrGrU rUrGrG rArU*mA* mU*mA |
| SEQ ID NO: | mG*mG*mC* rUrUrA rUrArU rCrCrA rArCrA rCrUrU rCrGrG rUrUrU rUrArG rArGrC |
| 25 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rArArG |
| | rGrArC rCrCrC rArCrG rArGrA rArUrU rCrArG rUrGrU rUrGrG rArU*mA* mU*mA |
| SEQ ID NO: | mA*mU*mU* rUrCrA rCrArU rArArA rArCrU rCrUrUrUrUrG rUrUrU rUrArG rArGrC |
| 26 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rUrUrA |
| | rArCrC rUrArA rArGrA rArUrU rCrArG rArGrU rUrUrU rArU*mG* mU*mG |
| SEQ ID NO: | mA*mU*mU* rUrCrA rCrArU rArArA rArCrU rCrUrUrUrUrG rUrUrU rUrArG rArGrC |
| 27 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rUrCrU |
| | rUrUrA rArCrC rUrArA rArGrA rArUrU rCrArG rArGrU rUrUrU rArU*mG* mU*mG |
| SEQ ID NO: | mA*mU*mU* rUrCrA rCrArU rArArA rArCrU rCrUrUrUrUrG rUrUrU rUrArG rArGrC |
| 28 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rUrUrA |
| | rArCrC rUrArA rArGrA rArUrU rCrArG rArGrU rUrUrU rArU*mG* mU*mG |
| SEQ ID NO: | mA*mU*mU* rUrCrA rCrArU rArArA rArCrU rCrUrUrUrUrG rUrUrU rUrArG rArGrC |
| 29 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rUrCrU |
| | rUrUrA rArCrC rUrArA rArGrA rArUrU rCrArG rArGrU rUrUrU rArU*mG* mU*mG |
| SEQ ID NO: | mC*mU*mU* rArGrA rGrArA rUrArU rUrUrG rUrArG rArGrG rUrUrU rUrArG rArGrC |
| 30 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rUrGrU |
| | rGrCrC rUrCrU rCrGrA rArUrU rCrUrA rCrArA rArUrA rUrU*mC* mU*mC |
| SEQ ID NO: | mC*mU*mU* rArGrA rGrArA rUrArU rUrUrG rUrArG rArGrG rUrUrU rUrArG rArGrC |
| 31 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rArArA |
| | rUrGrU rGrCrC rUrCrU rCrGrA rArUrU rCrUrA rCrArA rArUrA rUrU*mC* mU*mC |
| SEQ ID NO: | mC*mU*mU* rArGrA rGrArA rUrArU rUrUrG rUrArG rArGrG rUrUrU rUrArG rArGrC |
| 32 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rUrGrU |
| | rGrCrC rUrCrU rCrGrA rArUrU rCrUrA rCrArA rArUrA rUrU*mC* mU*mC |
| SEQ ID NO: | mC*mU*mU* rArGrA rGrArA rUrArU rUrUrG rUrArG rArGrG rUrUrU rUrArG rArGrC |
| 33 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rArArA |
| | rUrGrU rGrCrC rUrCrU rCrGrA rArUrU rCrUrA rCrArA rArUrA rUrU*mC* mU*mC |
| SEQ ID NO: | mU*mU*mG* rArCrU rArUrA rArUrG rArArU rArCrU rUrCrG rUrUrU rUrArG rArGrC |
| 34 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rArArU |
| | rCrCrC rUrGrA rArGrA rArUrU rCrGrU rArUrU rCrArU rUrA*mU* mA*mG |
| SEQ ID NO: | mU*mU*mG* rArCrU rArUrA rArUrG rArArU rArCrU rUrCrG rUrUrU rUrArG rArGrC |
| 35 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rUrCrA |
| | rArArU rCrCrC rUrGrA rArGrA rArUrU rCrGrU rArUrU rCrArU rUrA*mU* mA*mG |
| SEQ ID NO: | mU*mU*mG* rArCrU rArUrA rArUrG rArArU rArCrU rUrCrG rUrUrU rUrArG rArGrC |
| 36 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rArArU |
| | rCrCrC rUrGrA rArGrA rArUrU rCrGrU rArUrU rCrArU rUrA*mU* mA*mG |
| SEQ ID NO: | mU*mU*mG* rArCrU rArUrA rArUrG rArArU rArCrU rUrCrG rUrUrU rUrArG rArGrC |
| 37 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rUrCrA |
| | rArArU rCrCrC rUrGrA rArGrA rArUrU rCrGrU rArUrU rCrArU rUrA*mU* mA*mG |
| SEQ ID NO: | mC*mA*mA* rArArC rArCrG rCrArU rArArA rArArU rUrUrG rUrUrU rUrArG rArGrC rUrArG |
| 38 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rCrUrU rUrCrC |
| | rUrArA rArGrA rArUrU rCrUrU rUrUrU rArUrG rCrG*mU* mG*mU |
| SEQ ID NO: | mC*mA*mA* rArArC rArCrG rCrArU rArArA rArArU rUrUrG rUrUrU rUrArG rArGrC rUrArG |
| 39 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rUrCrU rCrUrU |
| | rUrCrC rUrArA rArGrA rArUrU rCrUrU rUrUrU rArUrG rCrG*mU* mG*mU |
| SEQ ID NO: | mC*mA*mA* rArArC rArCrG rCrArU rArArA rArArU rUrUrG rUrUrU rUrArG rArGrC rUrArG |
| 40 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrGrA rCrCrG rArGrU rCrGrG rUrCrC rCrUrU rUrCrC |
| | rUrArA rArGrA rArUrU rCrUrU rUrUrU rArUrG rCrG*mU* mG*mU |
| SEQ ID NO: | mC*mA*mA* rArArC rArCrG rCrArU rArArA rArArU rUrUrG rUrUrU rUrArG rArGrC rUrArG |
| 41 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrGrA rCrCrG rArGrU rCrGrG rUrCrC rUrCrU rCrUrU |
| | rUrCrC rUrArA rArGrA rArUrU rCrUrU rUrUrU rArUrG rCrG*mU* mG*mU |
| SEQ ID NO: | mU*mC*mA* rArArU rUrArU rGrArG rGrUrG rCrUrG rGrArG rUrUrU rUrArG rArGrC |
| 42 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rUrUrC |
| | rUrCrC rUrUrC rCrGrA rArUrU rCrArG rCrArC rCrUrC rArU*mA* mA*mU |
| SEQ ID NO: | mU*mC*mA* rArArU rUrArU rGrArG rGrUrG rCrUrG rGrArG rUrUrU rUrArG rArGrC |
| 43 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrUrU |
| | rUrUrC rUrCrC rUrUrC rCrGrA rArUrU rCrArG rCrArC rCrUrC rArU*mA* mA*mU |
| SEQ ID NO: | mU*mC*mA* rArArU rUrArU rGrArG rGrUrG rCrUrG rGrArG rUrUrU rUrArG rArGrC |
| 44 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rUrUrC |
| | rUrCrC rUrUrC rCrGrA rArUrU rCrArG rCrArC rCrUrC rArU*mA* mA*mU |
| SEQ ID NO: | mU*mC*mA* rArArU rUrArU rGrArG rGrUrG rCrUrG rGrArG rUrUrU rUrArG rArGrC |
| 45 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rGrUrU |
| | rUrUrC rUrCrC rUrUrC rCrGrA rArUrU rCrArG rCrArC rCrUrC rArU*mA* mA*mU |
| SEQ ID NO: | mU*mA*mC* rArGrC rUrUrU rArUrG rUrGrA rCrUrA rArUrG rUrUrU rUrArG rArGrC |
| 46 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | mU*mA*mC* rArGrC rUrUrU rArUrG rUrGrA rCrUrA rArUrG rUrUrU rUrArG rArGrC |
| 47 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrArU |
| | rGrGrU rUrCrC rCrArU rUrGrA rArUrU rCrArG rUrCrA rCrArU rArA*mA* mG*mC |
| SEQ ID NO: | mU*mA*mC* rArGrC rUrUrU rArUrG rUrGrA rCrUrA rArUrG rUrUrU rUrArG rArGrC |
| 48 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rGrGrU |
| | rUrCrC rCrArU rUrGrA rArUrU rCrArG rUrCrA rCrArU rArA*mA* mG*mC |
| SEQ ID NO: | mU*mA*mC* rArGrC rUrUrU rArUrG rUrGrA rCrUrA rArUrG rUrUrU rUrArG rArGrC |
| 49 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrGrA rCrCrG rArGrU rCrGrG rUrCrC rGrArU |
| | rGrGrU rUrCrC rCrArU rUrGrA rArUrU rCrArG rUrCrA rCrArU rArA*mA* mG*mC |
| SEQ ID NO: | mU*mA*mC* rArGrC rUrUrU rArUrG rUrGrA rCrUrA rArUrG rUrUrU rUrArG rArGrC |
| 50 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArArA rGrC |
| SEQ ID NO: | mU*mA*mC* rArGrC rUrUrU rArUrG rUrGrA rCrUrA rArUrG rUrUrU rUrArG rArGrC |
| 51 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArArA rGmC |
| SEQ ID NO: | mU*mA*mC* rArGrC rUrUrU rArUrG rUrGrA rCrUrA rArUrG rUrUrU rUrArG rArGrC |
| 52 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArArA mGmC |
| SEQ ID NO: | mU*mA*mC* rArGrC rUrUrU rArUrG rUrGrA rCrUrA rArUrG rUrUrU rUrArG rArGrC |
| 53 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArAmA mGmC |
| SEQ ID NO: | mU*mA*mC* rArGrC rUrUrU rArUrG rUrGrA rCrUrA rArUrG rUrUrU rUrArG rArGrC |
| 55 | rUrArG rArArA rUrArG rCrArA rGrUrU rArArA rArUrA rArGrG rCrUrA rGrUrC rCrGrU |
| | rUrArU rCrArA rCrUrU rGrArA rArArA rGrUrG rGrCrA rCrCrG rArGrU rCrGrG rUrGrC |
| | rGrGrU rUrCrC rCrArU rUrGrA rArUrU rCrArG rUrCrA rCrArU mAmAmA mGmC |
| SEQ ID NO: | mU*mA*mC* rArGrC rUrUrU rArUrG rUrGrA rCrUrA rArUrG rUrUrU rUrArG rArGrC |
| 56 | rUrArG rArArA rUrArG rCrArA rGrUrU rArArA rArUrA rArGrG rCrUrA rGrUrC rCrGrU |
| | rUrArU rCrArA rCrUrU rGrArA rArArA rGrUrG rGrCrA rCrCrG rArGrU rCrGrG rUrGrC |
| | rGrGrU rUrCrC rCrArU rUrGrA rArUrU rCrArG rUrCrA rCrAmU mAmAmA mGmC |
| SEQ ID NO: | mU*mA*mC* rArGrC rUrUrU rArUrG rUrGrA rCrUrA rArUrG rUrUrU rUrArG rArGrC |
| 57 | rUrArG rArArA rUrArG rCrArA rGrUrU rArArA rArUrA rArGrG rCrUrA rGrUrC rCrGrU |
| | rUrArU rCrArA rCrUrU rGrArA rArArA rGrUrG rGrCrA rCrCrG rArGrU rCrGrG rUrGrC |
| | rGrGrU rUrCrC rCrArU rUrGrA rArUrU rCrArG rUrCrA rCmAmU mAmAmA mGmC |
| SEQ ID NO: | mU*mA*mC* rArGrC rUrUrU rArUrG rUrGrA rCrUrA rArUrG rUrUrU rUrArG rArGrC |
| 58 | rUrArG rArArA rUrArG rCrArA rGrUrU rArArA rArUrA rArGrG rCrUrA rGrUrC rCrGrU |
| | rUrArU rCrArA rCrUrU rGrArA rArArA rGrUrG rGrCrA rCrCrG rArGrU rCrGrG rUrGrC |
| | rGrGrU rUrCrC rCrArU rUrGrA rArUrU rCrArG rUrCrA mCmAmU mAmAmA mGmC |
| SEQ ID NO: | mU*mA*mC* rArGrC rUrUrU rArUrG rUrGrA rCrUrA rArUrG rUrUrU rUrArG rArGrC |
| 59 | rUrArG rArArA rUrArG rCrArA rGrUrU rArArA rArUrA rArGrG rCrUrA rGrUrC rCrGrU |
| | rUrArU rCrArA rCrUrU rGrArA rArArA rGrUrG rGrCrA rCrCrG rArGrU rCrGrG rUrGrC |
| | rGrGrU rUrCrC rCrArU rUrGrA rArUrU rCrArG rUrCmA mCmAmU mAmAmA mGmC |
| SEQ ID NO: | mU*mA*mC* rArGrC rUrUrU rArUrG rUrGrA rCrUrA rArUrG rUrUrU rUrArG rArGrC |
| 60 | rUrArG rArArA rUrArG rCrArA rGrUrU rArArA rArUrA rArGrG rCrUrA rGrUrC rCrGrU |
| | rUrArU rCrArA rCrUrU rGrArA rArArA rGrUrG rGrCrA rCrCrG rArGrU rCrGrG rUrGrC |
| | rGrGrU rUrCrC rCrArU rUrGrA rArUrU rCrArG rUmCmA mCmAmU mAmAmA mGmC |
| SEQ ID NO: | mU*mA*mC* rArGrC rUrUrU rArUrG rUrGrA rCrUrA rArUrG rUrUrU rUrArG rArGrC |
| 61 | rUrArG rArArA rUrArG rCrArA rGrUrU rArArA rArUrA rArGrG rCrUrA rGrUrC rCrGrU |
| | rUrArU rCrArA rCrUrU rGrArA rArArA rGrUrG rGrCrA rCrCrG rArGrU rCrGrG rUrGrC |
| | rGrGrU rUrCrC rCrArU rUrGrA rArUrU rCrArG rUrCrA rCrArU rArArA rG*rC |
| SEQ ID NO: | mU*mA*mC* rArGrC rUrUrU rArUrG rUrGrA rCrUrA rArUrG rUrUrU rUrArG rArGrC |
| 62 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArArA* rG*rC |
| SEQ ID NO: | mU*mA*mC* rArGrC rUrUrU rArUrG rUrGrA rCrUrA rArUrG rUrUrU rUrArG rArGrC |
| 63 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*rA* rG*rC |
| SEQ ID NO: | mU*mA*mC* rArGrC rUrUrU rArUrG rUrGrA rCrUrA rArUrG rUrUrU rUrArG rArGrC |
| 64 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rA*rA*rA* rG*rC |
| SEQ ID NO: | mU*mA*mC* rArGrC rUrUrU rArUrG rUrGrA rCrUrA rArUrG rUrUrU rUrArG rArGrC |
| 65 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU* rA*rA*rA* rG*rC |
| SEQ ID NO: | mU*mA*mC* rArGrC rUrUrU rArUrG rUrGrA rCrUrA rArUrG rUrUrU rUrArG rArGrC |
| 66 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrA*rU* rA*rA*rA* rG*rC |
| SEQ ID NO: | mU*mA*mC* rArGrC rUrUrU rArUrG rUrGrA rCrUrA rArUrG rUrUrU rUrArG rArGrC |
| 67 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rC*rA*rU* rA*rA*rA* rG*rC |
| SEQ ID NO: | mU*mA*mC* rArGrC rUrUrU rArUrG rUrGrA rCrUrA rArUrG rUrUrU rUrArG rArGrC |
| 68 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA* rC*rA*rU* rA*rA*rA* rG*rC |
| SEQ ID NO: | mU*mA*mC* rArGrC rUrUrU rArUrG rUrGrA rCrUrA rArUrG rUrUrU rUrArG rArGrC |
| 69 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrC*rA* rC*rA*rU* rA*rA*rA* rG*rC |
| SEQ ID NO: | mU*mA*mC* rArGrC rUrUrU rArUrG rUrGrA rCrUrA rArUrG rUrUrU rUrArG rArGrC |
| 70 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rU*rC*rA* rC*rA*rU* rA*rA*rA* rG*rC |
| SEQ ID NO: | mU*mA*mC* rArGrC rUrUrU rArUrG rUrGrA rCrUrA rArUrG rUrUrU rUrArG rArGrC |
| 71 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArArA rG*mC |
| SEQ ID NO: | mU*mA*mC* rArGrC rUrUrU rArUrG rUrGrA rCrUrA rArUrG rUrUrU rUrArG rArGrC |
| 72 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArArA* mG*mC |
| SEQ ID NO: | mU*mA*mC* rArGrC rUrUrU rArUrG rUrGrA rCrUrA rArUrG rUrUrU rUrArG rArGrC |
| 73 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | mU*mA*mC* rArGrC rUrUrU rArUrG rUrGrA rCrUrA rArUrG rUrUrU rUrArG rArGrC |
| 74 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rA*mA*mA* mG*mC |
| SEQ ID NO: | mU*mA*mC* rArGrC rUrUrU rArUrG rUrGrA rCrUrA rArUrG rUrUrU rUrArG rArGrC |
| 75 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU* mA*mA*mA* mG*mC |
| SEQ ID NO: | mU*mA*mC* rArGrC rUrUrU rArUrG rUrGrA rCrUrA rArUrG rUrUrU rUrArG rArGrC |
| 76 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrA*mU* mA*mA*mA* mG*mC |
| SEQ ID NO: | mU*mA*mC* rArGrC rUrUrU rArUrG rUrGrA rCrUrA rArUrG rUrUrU rUrArG rArGrC |
| 77 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rC*mA*mU* mA*mA*mA* mG*mC |
| SEQ ID NO: | mU*mA*mC* rArGrC rUrUrU rArUrG rUrGrA rCrUrA rArUrG rUrUrU rUrArG rArGrC |
| 78 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA* mC*mA*mU* mA*mA*mA* mG*mC |
| SEQ ID NO: | mU*mA*mC* rArGrC rUrUrU rArUrG rUrGrA rCrUrA rArUrG rUrUrU rUrArG rArGrC |
| 79 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrC*mA* mC*mA*mU* mA*mA*mA* mG*mC |
| SEQ ID NO: | mU*mA*mC* rArGrC rUrUrU rArUrG rUrGrA rCrUrA rArUrG rUrUrU rUrArG rArGrC |
| 80 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rU*mC*mA* mC*mA*mU* mA*mA*mA* |
| | mG*mC |
| SEQ ID NO: | mU*mA*mC* rArGrC rUrUrU rArUrG rUrGrA rCrUrA rArUrG rUrUrU rUrArG rArGrC |
| 81 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rAmA*mA* mG*rC |
| SEQ ID NO: | rUrArC rArGrC rUrUrU rArUrG rUrGrA rCrUrA rArUrG rUrUrU rUrArG rArGrC rUrArG |
| 82 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rGrGrU rUrCrC |
| | rCrArU rUrGrA rArUrU rCrArG rUrCrA rCrArU rArA*mA* mG*mC |
| SEQ ID NO: | mUrArC rArGrC rUrUrU rArUrG rUrGrA rCrUrA rArUrG rUrUrU rUrArG rArGrC rUrArG |
| 83 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rGrGrU rUrCrC |
| | rCrArU rUrGrA rArUrU rCrArG rUrCrA rCrArU rArA*mA* mG*mC |
| SEQ ID NO: | mUmArC rArGrC rUrUrU rArUrG rUrGrA rCrUrA rArUrG rUrUrU rUrArG rArGrC rUrArG |
| 84 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rGrGrU rUrCrC |
| | rCrArU rUrGrA rArUrU rCrArG rUrCrA rCrArU rArA*mA* mG*mC |
| SEQ ID NO: | mUmAmC mArGrC rUrUrU rArUrG rUrGrA rCrUrA rArUrG rUrUrU rUrArG rArGrC rUrArG |
| 85 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rGrGrU rUrCrC |
| | rCrArU rUrGrA rArUrU rCrArG rUrCrA rCrArU rArA*mA* mG*mC |
| SEQ ID NO: | mUmAmC mAmGrC rUrUrU rArUrG rUrGrA rCrUrA rArUrG rUrUrU rUrArG rArGrC rUrArG |
| 86 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rGrGrU rUrCrC |
| | rCrArU rUrGrA rArUrU rCrArG rUrCrA rCrArU rArA*mA* mG*mC |
| SEQ ID NO: | mUmAmC mAmGmC rUrUrU rArUrG rUrGrA rCrUrA rArUrG rUrUrU rUrArG rArGrC |
| 87 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | mUmAmC mAmGmC mUrUrU rArUrG rUrGrA rCrUrA rArUrG rUrUrU rUrArG rArGrC |
| 88 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | mUmAmC mAmGmC mUmUrU rArUrG rUrGrA rCrUrA rArUrG rUrUrU rUrArG rArGrC |
| 89 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | mUmAmC mAmGmC mUmUmU rArUrG rUrGrA rCrUrA rArUrG rUrUrU rUrArG rArGrC |
| 90 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrGrUrGrC rGrGrU |
| | rUrCrC rCrArU rUrGrA rArUrU rCrArG rUrCrA rCrArU rArA*mA* mG*mC |
| SEQ ID NO: | mUmAmC mAmGmC mUmUmU mArUrG rUrGrA rCrUrA rArUrG rUrUrU rUrArG rArGrC |
| 91 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | rU*rArC rArGrC rUrUrU rArUrG rUrGrA rCrUrA rArUrG rUrUrU rUrArG rArGrC rUrArG |
| 92 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rGrGrU rUrCrC |
| | rCrArU rUrGrA rArUrU rCrArG rUrCrA rCrArU rArA*mA* mG*mC |
| SEQ ID NO: | rU*rA*rC rArGrC rUrUrU rArUrG rUrGrA rCrUrA rArUrG rUrUrU rUrArG rArGrC rUrArG |
| 93 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rGrGrU rUrCrC |
| | rCrArU rUrGrA rArUrU rCrArG rUrCrA rCrArU rArA*mA* mG*mC |
| SEQ ID NO: | rU*rA*rC* rArGrC rUrUrU rArUrG rUrGrA rCrUrA rArUrG rUrUrU rUrArG rArGrC rUrArG |
| 94 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rGrGrU rUrCrC |
| | rCrArU rUrGrA rArUrU rCrArG rUrCrA rCrArU rArA*mA* mG*mC |
| SEQ ID NO: | rU*rA*rC* rA*rGrC rUrUrU rArUrG rUrGrA rCrUrA rArUrG rUrUrU rUrArG rArGrC rUrArG |
| 95 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rGrGrU rUrCrC |
| | rCrArU rUrGrA rArUrU rCrArG rUrCrA rCrArU rArA*mA* mG*mC |
| SEQ ID NO: | rU*rA*rC* rA*rG*rC rUrUrU rArUrG rUrGrA rCrUrA rArUrG rUrUrU rUrArG rArGrC rUrArG |
| 96 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rGrGrU rUrCrC |
| | rCrArU rUrGrA rArUrU rCrArG rUrCrA rCrArU rArA*mA* mG*mC |
| SEQ ID NO: | rU*rA*rC* rA*rG*rC* rUrUrU rArUrG rUrGrA rCrUrA rArUrG rUrUrU rUrArG rArGrC |
| 97 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | rU*rA*rC* rA*rG*rC* rU*rUrU rArUrG rUrGrA rCrUrA rArUrG rUrUrU rUrArG rArGrC |
| 98 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | rU*rA*rC* rA*rG*rC* rU*rU*rU rArUrG rUrGrA rCrUrA rArUrG rUrUrU rUrArG rArGrC |
| 99 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | rU*rA*rC* rA*rG*rC* rU*rU*rU* rArUrG rUrGrA rCrUrA rArUrG rUrUrU rUrArG rArGrC |
| 100 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | rU*rA*rC* rA*rG*rC* rU*rU*rU* rA*rUrG rUrGrA rCrUrA rArUrG rUrUrU rUrArG rArGrC |
| 101 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | mU*rArC rArGrC rUrUrU rArUrG rUrGrA rCrUrA rArUrG rUrUrU rUrArG rArGrC rUrArG |
| 102 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rGrGrU rUrCrC |
| | rCrArU rUrGrA rArUrU rCrArG rUrCrA rCrArU rArA*mA* mG*mC |
| SEQ ID NO: | mU*mA*rC rArGrC rUrUrU rArUrG rUrGrA rCrUrA rArUrG rUrUrU rUrArG rArGrC rUrArG |
| 103 | rArArA rUrArG rCrArA rGrUrU rArArA rArUrA rArGrG rCrUrA rGrUrC rCrGrU rUrArU rCrArA |
| | rCrUrU rGrArA rArArA rGrUrG rGrCrA rCrCrG rArGrU rCrGrG rUrGrC rGrGrU rUrCrC |
| | rCrArU rUrGrA rArUrU rCrArG rUrCrA rCrArU rArA*mA* mG*mC |
| SEQ ID NO: | mU*mA*mC* rArGrC rUrUrU rArUrG rUrGrA rCrUrA rArUrG rUrUrU rUrArG rArGrC |
| 104 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | mU*mA*mC* mA*rGrC rUrUrU rArUrG rUrGrA rCrUrA rArUrG rUrUrU rUrArG rArGrC |
| 105 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | mU*mA*mC* mA*mG*rC rUrUrU rArUrG rUrGrA rCrUrA rArUrG rUrUrU rUrArG rArGrC |
| 106 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCrArG rUrCrA rCrArU rArA*mA* mG*mC |
| SEQ ID NO: | mU*mA*mC* mA*mG*mC* rUrUrU rArUrG rUrGrA rCrUrA rArUrG rUrUrU rUrArG |
| 107 | rArGrC rUrArG rArArA rUrArG rCrArA rGrUrU rArArA rArUrA rArGrG rCrUrA rGrUrC rCrGrU |
| | rUrArU rCrArA rCrUrU rGrArA rArArA rGrUrG rGrCrA rCrCrG rArGrU rCrGrG rUrGrC |
| | rGrGrU rUrCrC rCrArU rUrGrA rArUrU rCrArG rUrCrA rCrArU rArA*mA* mG*mC |
| SEQ ID NO: | mU*mA*mC* mA*mG*mC* mU*rUrU rArUrG rUrGrA rCrUrA rArUrG rUrUrU rUrArG |
| 108 | rArGrC rUrArG rArArA rUrArG rCrArA rGrUrU rArArA rArUrA rArGrG rCrUrA rGrUrC rCrGrU |
| | rUrArU rCrArA rCrUrU rGrArA rArArA rGrUrG rGrCrA rCrCrG rArGrU rCrGrG rUrGrC |
| | rGrGrU rUrCrC rCrArU rUrGrA rArUrU rCrArG rUrCrA rCrArU rArA*mA* mG*mC |
| SEQ ID NO: | mU*mA*mC* mA*mG*mC* mU*mU*rU rArUrG rUrGrA rCrUrA rArUrG rUrUrU rUrArG |
| 109 | rArGrC rUrArG rArArA rUrArG rCrArA rGrUrU rArArA rArUrA rArGrG rCrUrA rGrUrC rCrGrU |
| | rUrArU rCrArA rCrUrU rGrArA rArArA rGrUrG rGrCrA rCrCrG rArGrU rCrGrG rUrGrC |
| | rGrGrU rUrCrC rCrArU rUrGrA rArUrU rCrArG rUrCrA rCrArU rArA*mA* mG*mC |
| SEQ ID NO: | mU*mA*mC* mA*mG*mC* mU*mU*mU* rArUrG rUrGrA rCrUrA rArUrG rUrUrU |
| 110 | rUrArG rArGrC rUrArG rArArA rUrArG rCrArA rGrUrU rArArA rArUrA rArGrG rCrUrA rGrUrC |
| | rCrGrU rUrArU rCrArA rCrUrU rGrArA rArArA rGrUrG rGrCrA rCrCrG rArGrU rCrGrG |
| | rUrGrC rGrGrU rUrCrC rCrArU rUrGrA rArUrU rCrArG rUrCrA rCrArU rArA*mA* mG*mC |
| SEQ ID NO: | mU*mA*mC* mA*mG*mC* mU*mU*mU* mA*rUrGrUrGrA rCrUrA rArUrG rUrUrU |
| 111 | rUrArG rArGrC rUrArG rArArA rUrArG rCrArA rGrUrU rArArA rArUrA rArGrG rCrUrA rGrUrC |
| | rCrGrU rUrArU rCrArA rCrUrU rGrArA rArArA rGrUrG rGrCrA rCrCrG rArGrU rCrGrG |
| | rUrGrC rGrGrU rUrCrC rCrArU rUrGrA rArUrU rCrArG rUrCrA rCrArU rArA*mA* mG*mC |
| SEQ ID NO: | mU*mA*mC* rArGrC rUrUrU rArUrG rUrGrA rCrUrA rArUrG rUrUrU rUrArG rArGrC |
| 112 | rUrArG rArArA rUrArG rCrArA rGrUrU rArArA rArUrA rArGrG rCrUrA rGrUrC rCrGrU rUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rCmAmG mUmCmA mCmAmU mAmA*mA* mG*mC |
| SEQ ID NO: | mU*mA*mC* rArGrC rUrUrU rArUrG rUrGrA rCrUrA rArUrG rUrUrU rUrArG rArGrC |
| 113 | rUrArG rArArA rUrArG rCrArA rGrUrU rArArA rArUrA rArGrG rCrUrA rGrUrC rCrGrUrUrArU |
| | rCrArA rCrUrU rGrArA rArArA rGrUrG rGrCrA rCrCrG rArGrU rCrGrG rUrGrC rGrGrU |
| | rUrCrC rCrArU rUrGrA rArUrU rC*mA*mG* mU*mC*mA* mC*mA*mU* mA*mA*mA* |
| | mG*mC |
| SEQ ID NO: | mA*mA*mU*rUrArUrGrGrGrGrArUrUrArCrUrArGrGrArGrUrUrUrUrArGrArGrCrUrAr |
| 114 | GrArArArUrArGrCrArArGrUrUrArArArArUrArArGrGrCrUrArGrUrCrCrGrUrUrArUrCrAr |
| | ArCrUrUrGrArArArArArGrUrGrGrCrArCrCrGrArGrUrCrGrGrUrGrCrUrGrCrCrCrCrUrUrC |
| | rCrGrArArUrUrCrUrArGrUrArA*mU*mC*mC*mC*mC*mA*mU |
| SEQ ID NO: | mU*mC*mC*rArUrUrUrCrArUrArGrUrCrUrUrUrCrCrUrGrUrUrUrUrArGrArGrCrUrArG |
| 115 | rArArArUrArGrCrArArGrUrUrArArArArUrArArGrGrCrUrArGrUrCrCrGrUrUrArUrCrArAr |
| | CrUrUrGrArArArArArGrUrGrGrCrArCrCrGrArGrUrCrGrGrUrGrCrArCrArCrCrCrArArGrG |
| | rGrArArUrUrCrArArArGrArC*mU*mA*mU*mG*mA*mA*mA |
| SEQ ID NO: | mG*mG*mU*rCrArCrUrUrUrUrArArCrArCrArCrCrCrArGrUrUrUrUrArGrArGrCrUrArGr |
| 116 | ArArArUrArGrCrArArGrUrUrArArArArUrArArGrGrCrUrArGrUrCrCrGrUrUrArUrCrArAr |
| | CrUrUrGrArArArArArGrUrGrGrCrArCrCrGrArGrUrCrGrGrUrGrCrCrUrUrUrCrCrUrUrGr |
| | GrGrArArUrUrCrGrUrGrUrGrU*mU*mA*mA*mA*mA*mG*mU |
| SEQ ID NO: | mU*mU*mU*rUrGrUrArArUrUrArArCrArGrCrUrUrGrCrGrUrUrUrUrArGrArGrCrUrArG |
| 117 | rArArArUrArGrCrArArGrUrUrArArArArUrArArGrGrCrUrArGrUrCrCrGrUrUrArUrCrArAr |
| | CrUrUrGrArArArArArGrUrGrGrCrArCrCrGrArGrUrCrGrGrUrGrCrUrUrCrArCrCrArGrCrA |
| | rGrArArUrUrCrArGrCrUrGrU*mU*mA*mA*mU*mU*mA*mC |
| SEQ ID NO: | mC*mU*mU*rArUrArUrCrCrArArCrArCrUrUrCrGrUrGrGrUrUrUrUrArGrArGrCrUrArGr |
| 118 | ArArArUrArGrCrArArGrUrUrArArArArUrArArGrGrCrUrArGrUrCrCrGrUrUrArUrCrArAr |
| | CrUrUrGrArArArArArGrUrGrGrCrArCrCrGrArGrUrCrGrGrUrGrCrArGrGrArCrCrCrCrArC |
| | rGrArArUrUrCrGrArArGrUrG*mU*mU*mG*mG*mA*mU*mA |
| SEQ ID NO: | mG*mG*mC*rUrUrArUrArUrCrCrArArCrArCrUrUrCrGrGrUrUrUrUrArGrArGrCrUrArGrAr |
| 119 | ArArUrArGrCrArArGrUrUrArArArArUrArArGrGrCrUrArGrUrCrCrGrUrUrArUrCrArArCrUrUr |
| | GrArArArArArGrUrGrGrCrArCrCrGrArGrUrCrGrGrUrGrCrGrArCrCrCrCrArCrGrArGrArArU |
| | rUrCrArGrUrGrUrU*mG*mG*mA*mU*mA*mU*mA |
| SEQ ID NO: | mA*mU*mU*rUrCrArCrArUrArArArArCrUrCrUrUrUrUrGrUrUrUrUrArGrArGrCrUrArGrAr |
| 120 | ArArUrArGrCrArArGrUrUrArArArArUrArArGrGrCrUrArGrUrCrCrGrUrUrArUrCrArArCrUrUr |
| | GrArArArArArGrUrGrGrCrArCrCrGrArGrUrCrGrGrUrGrCrUrUrArArCrCrUrArArArGrArArUr |
| | UrCrArGrArGrUrU*mU*mU*mA*mU*mG*mU*mG |
| SEQ ID NO: | mC*mU*mU*rArGrArGrArArUrArUrUrUrGrUrArGrArGrGrUrUrUrUrArGrArGrCrUrArGrAr |
| 121 | ArArUrArGrCrArArGrUrUrArArArArUrArArGrGrCrUrArGrUrCrCrGrUrUrArUrCrArArCrUrUr |
| | GrArArArArArGrUrGrGrCrArCrCrGrArGrUrCrGrGrUrGrCrUrGrUrGrCrCrUrCrUrCrGrArAr |
| | UrUrCrUrArCrArArA*mU*mA*mU*mU*mC*mU*mC |
| SEQ ID NO: | mU*mU*mG*rArCrUrArUrArArUrGrArArUrArCrUrUrCrGrUrUrUrUrArGrArGrCrUrArGrAr |
| 122 | ArArUrArGrCrArArGrUrUrArArArArUrArArGrGrCrUrArGrUrCrCrGrUrUrArUrCrArArCrUrUr |
| | GrArArArArArGrUrGrGrCrArCrCrGrArGrUrCrGrGrUrGrCrArArUrCrCrCrUrGrArArGrArArU |
| | rUrCrGrUrArUrUrC*mA*mU*mU*mA*mU*mA*mG |
| SEQ ID NO: | mC*mA*mA*rArArCrArCrGrCrArUrArArArArArUrUrUrGrUrUrUrUrArGrArGrCrUrArGrArA |
| 123 | rArUrArGrCrArArGrUrUrArArArArUrArArGrGrCrUrArGrUrCrCrGrUrUrArUrCrArArCrUrUrG |
| | rArArArArArGrUrGrGrCrArCrCrGrArGrUrCrGrGrUrGrCrCrUrUrUrCrCrUrArArArGrArArUr |
| | UrCrUrUrUrUrUrA*mU*mG*mC*mG*mU*mG*mU |
| SEQ ID NO: | mU*mC*mA*rArArUrUrArUrGrArGrGrUrGrCrUrGrGrArGrUrUrUrUrArGrArGrCrUrArGrAr |
| 124 | ArArUrArGrCrArArGrUrUrArArArArUrArArGrGrCrUrArGrUrCrCrGrUrUrArUrCrArArCrUrUr |
| | GrArArArArArGrUrGrGrCrArCrCrGrArGrUrCrGrGrUrGrCrUrUrCrUrCrCrUrUrCrCrGrArAr |
| | UrUrCrArGrCrArCrC*mU*mC*mA*mU*mA*mA*mU |
| SEQ ID NO: | mU*mA*mC*rArGrCrUrUrUrArUrGrUrGrArCrUrArArUrGrUrUrUrUrArGrArGrCrUrArGrAr |
| 125 | ArArUrArGrCrArArGrUrUrArArArArUrArArGrGrCrUrArGrUrCrCrGrUrUrArUrCrArArCrUrUr |
| | GrArArArArArGrUrGrGrCrArCrCrGrArGrUrCrGrGrUrGrCrGrGrUrUrCrCrCrArUrUrGrArArU |
| | rUrCrArGrUrCrArC*mA*mU*mA*mA*mA*mG*mC |
| SEQ ID NO: | mA*mA*rUrUrArUrGrGrGrGrArUrUrArCrUrArGrGrArGrUrUrUrUrArGrArGrCrUrArGrArAr |
| 126 | ArUrArGrCrArArGrUrUrArArArArUrArArGrGrCrUrArGrUrCrCrGrUrUrArUrCrArArCrUrUrGr |
| | ArArArArArGrUrGrGrCrArCrCrGrArGrUrCrGrGrUrGrCrUrGrCrCrCrCrUrUrCrCrGrArArUr |
| | UrCrUrArGrUrArA*mU*mC*mC*mC*mC*mA*mU |
| SEQ ID NO: | mU*mC*rCrArUrUrUrCrArUrArGrUrCrUrUrUrCrCrUrGrUrUrUrUrArGrArGrCrUrArGrArAr |
| 127 | ArUrArGrCrArArGrUrUrArArArArUrArArGrGrCrUrArGrUrCrCrGrUrUrArUrCrArArCrUrUrGr |
| | ArArArArArGrUrGrGrCrArCrCrGrArGrUrCrGrGrUrGrCrArCrArCrCrCrArArGrGrGrArArUrU |
| | rCrArArArGrArC*mU*mA*mU*mG*mA*mA*mA |
| SEQ ID NO: | mG*mG*rUrCrArCrUrUrUrUrArArCrArCrArCrCrCrArGrUrUrUrUrArGrArGrCrUrArGrArAr |
| 128 | ArUrArGrCrArArGrUrUrArArArArUrArArGrGrCrUrArGrUrCrCrGrUrUrArUrCrArArCrUrUrGr |
| | ArArArArArGrUrGrGrCrArCrCrGrArGrUrCrGrGrUrGrCrCrUrUrUrCrCrUrUrGrGrGrArArUr |
| | UrCrGrUrGrUrGrU*mU*mA*mA*mA*mA*mG*mU |
| SEQ ID NO: | mU*mU*rUrUrGrUrArArUrUrArArCrArGrCrUrUrGrCrGrUrUrUrUrArGrArGrCrUrArGrArAr |
| 129 | ArUrArGrCrArArGrUrUrArArArArUrArArGrGrCrUrArGrUrCrCrGrUrUrArUrCrArArCrUrUrGr |
| | ArArArArArGrUrGrGrCrArCrCrGrArGrUrCrGrGrUrGrCrUrUrCrArCrCrArGrCrArGrArArUrU |
| | rCrArGrCrUrGrU*mU*mA*mA*mU*mU*mA*mC |
| SEQ ID NO: | mC*mU*rUrArUrArUrCrCrArArCrArCrUrUrCrGrUrGrGrUrUrUrUrArGrArGrCrUrArGrArAr |
| 130 | ArUrArGrCrArArGrUrUrArArArArUrArArGrGrCrUrArGrUrCrCrGrUrUrArUrCrArArCrUrUrGr |
| | ArArArArArGrUrGrGrCrArCrCrGrArGrUrCrGrGrUrGrCrArGrGrArCrCrCrCrArCrGrArArUrU |
| | rCrGrArArGrUrG*mU*mU*mG*mG*mA*mU*mA |
| SEQ ID NO: | mG*mG*rCrUrUrArUrArUrCrCrArArCrArCrUrUrCrGrGrUrUrUrUrArGrArGrCrUrArGrArAr |
| 131 | ArUrArGrCrArArGrUrUrArArArArUrArArGrGrCrUrArGrUrCrCrGrUrUrArUrCrArArCrUrUrGr |
| | ArArArArArGrUrGrGrCrArCrCrGrArGrUrCrGrGrUrGrCrGrArCrCrCrCrArCrGrArGrArArUrU |
| | rCrArGrUrGrUrU*mG*mG*mA*mU*mA*mU*mA |
| SEQ ID NO: | mA*mU*rUrUrCrArCrArUrArArArArCrUrCrUrUrUrUrGrUrUrUrUrArGrArGrCrUrArGrArArA |
| 132 | rUrArGrCrArArGrUrUrArArArArUrArArGrGrCrUrArGrUrCrCrGrUrUrArUrCrArArCrUrUrGrA |
| | rArArArArGrUrGrGrCrArCrCrGrArGrUrCrGrGrUrGrCrUrUrArArCrCrUrArArArGrArArUrUrC |
| | rArGrArGrUrU*mU*mU*mA*mU*mG*mU*mG |
| SEQ ID NO: | mC*mU*rUrArGrArGrArArUrArUrUrUrGrUrArGrArGrGrUrUrUrUrArGrArGrCrUrArGrArArA |
| 133 | rUrArGrCrArArGrUrUrArArArArUrArArGrGrCrUrArGrUrCrCrGrUrUrArUrCrArArCrUrUrGrA |
| | rArArArArGrUrGrGrCrArCrCrGrArGrUrCrGrGrUrGrCrUrGrUrGrCrCrUrCrUrCrGrArArUrUr |
| | CrUrArCrArArA*mU*mA*mU*mU*mC*mU*mC |
| SEQ ID NO: | mU*mU*rGrArCrUrArUrArArUrGrArArUrArCrUrUrCrGrUrUrUrUrArGrArGrCrUrArGrArArA |
| 134 | rUrArGrCrArArGrUrUrArArArArUrArArGrGrCrUrArGrUrCrCrGrUrUrArUrCrArArCrUrUrGrA |
| | rArArArArGrUrGrGrCrArCrCrGrArGrUrCrGrGrUrGrCrArArUrCrCrCrUrGrArArGrArArUrUr |
| | CrGrUrArUrUrC*mA*mU*mU*mA*mU*mA*mG |
| SEQ ID NO: | mC*mA*rArArArCrArCrGrCrArUrArArArArArUrUrUrGrUrUrUrUrArGrArGrCrUrArGrArArAr |
| 135 | UrArGrCrArArGrUrUrArArArArUrArArGrGrCrUrArGrUrCrCrGrUrUrArUrCrArArCrUrUrGrAr |
| | ArArArArGrUrGrGrCrArCrCrGrArGrUrCrGrGrUrGrCrCrUrUrUrCrCrUrArArArGrArArUrUrC |
| | rUrUrUrUrUrA*mU*mG*mC*mG*mU*mG*mU |
| SEQ ID NO: | mU*mC*rArArArUrUrArUrGrArGrGrUrGrCrUrGrGrArGrUrUrUrUrArGrArGrCrUrArGrArAr |
| 136 | ArUrArGrCrArArGrUrUrArArArArUrArArGrGrCrUrArGrUrCrCrGrUrUrArUrCrArArCrUrUrGr |
| | ArArArArArGrUrGrGrCrArCrCrGrArGrUrCrGrGrUrGrCrUrUrCrUrCrCrUrUrCrCrGrArArUr |
| | UrCrArGrCrArCrC*mU*mC*mA*mU*mA*mA*mU |
| SEQ ID NO: | mU*mA*rCrArGrCrUrUrUrArUrGrUrGrArCrUrArArUrGrUrUrUrUrArGrArGrCrUrArGrArAr |
| 137 | ArUrArGrCrArArGrUrUrArArArArUrArArGrGrCrUrArGrUrCrCrGrUrUrArUrCrArArCrUrUrGr |
| | ArArArArArGrUrGrGrCrArCrCrGrArGrUrCrGrGrUrGrCrGrGrUrUrCrCrCrArUrUrGrArArUrU |
| | rCrArGrUrCrArC*mA*mU*mA*mA*mA*mG*mC |
| SEQ ID NO: | mU*mA*mC*rArGrCrUrUrUrArUrGrUrGrArCrUrArArUrGrUrUrUrUrArGrArGrCrUrArGrAr |
| 138 | ArArUrArGrCrArArGrUrUrArArArArUrArArGrGrCrUrArGrUrCrCrGrUrUrArUrCrArArCrUrUr |
| | GrArArArArArGrUrGrGrCrArCrCrGrArGrUrCrGrGrUrGrCrGrGrUrUrCrCrCrArUrUrGrArArU |
| | rUrCrArGrUrCrArCrArUrArArArGC |
| SEQ ID NO: | mU*mA*mC*rArGrCrUrUrUrArUrGrUrGrArCrUrArArUrGrUrUrUrUrArGrArGrCrUrArGrAr |
| 139 | ArArUrArGrCrArArGrUrUrArArArArUrArArGrGrCrUrArGrUrCrCrGrUrUrArUrCrArArCrUrUr |
| | GrArArArArArGrUrGrGrCrArCrCrGrArGrUrCrGrGrUrGrCrGrGrUrUrCrCrCrArUrUrGrArArU |
| | rUrCrArGrUrCrArCrArUrArArAGC |
| SEQ ID NO: | mU*mA*mC*rArGrCrUrUrUrArUrGrUrGrArCrUrArArUrGrUrUrUrUrArGrArGrCrUrArGrAr |
| 140 | ArArUrArGrCrArArGrUrUrArArArArUrArArGrGrCrUrArGrUrCrCrGrUrUrArUrCrArArCrUrUr |
| | GrArArArArArGrUrGrGrCrArCrCrGrArGrUrCrGrGrUrGrCrGrGrUrUrCrCrCrArUrUrGrArArU |
| | rUrCrArGrUrCrArCrArUrArAAGC |
| SEQ ID NO: | mU*mA*mC*rArGrCrUrUrUrArUrGrUrGrArCrUrArArUrGrUrUrUrUrArGrArGrCrUrArGrAr |
| 141 | ArArUrArGrCrArArGrUrUrArArArArUrArArGrGrCrUrArGrUrCrCrGrUrUrArUrCrArArCrUrUr |
| | GrArArArArArGrUrGrGrCrArCrCrGrArGrUrCrGrGrUrGrCrGrGrUrUrCrCrCrArUrUrGrArArU |
| | rUrCrArGrUrCrArCrArUrAAAGC |
| SEQ ID NO: | mU*mA*mC*rArGrCrUrUrUrArUrGrUrGrArCrUrArArUrGrUrUrUrUrArGrArGrCrUrArGrAr |
| 142 | ArArUrArGrCrArArGrUrUrArArArArUrArArGrGrCrUrArGrUrCrCrGrUrUrArUrCrArArCrUrUr |
| | GrArArArArArGrUrGrGrCrArCrCrGrArGrUrCrGrGrUrGrCrGrGrUrUrCrCrCrArUrUrGrArArU |
| | rUrCrArGrUrCrArCrArUAAAGC |
| SEQ ID NO: | mU*mA*mC*rArGrCrUrUrUrArUrGrUrGrArCrUrArArUrGrUrUrUrUrArGrArGrCrUrArGrAr |
| 143 | ArArUrArGrCrArArGrUrUrArArArArUrArArGrGrCrUrArGrUrCrCrGrUrUrArUrCrArArCrUrUr |
| | GrArArArArArGrUrGrGrCrArCrCrGrArGrUrCrGrGrUrGrCrGrGrUrUrCrCrCrArUrUrGrArArU |
| | rUrCrArGrUrCrArCrATAAAGC |
| SEQ ID NO: | mU*mA*rCrArGrCrUrUrUrArUrGrUrGrArCrUrArArUrGrUrUrUrUrArGrArGrCrUrArGrArAr |
| 144 | ArUrArGrCrArArGrUrUrArArArArUrArArGrGrCrUrArGrUrCrCrGrUrUrArUrCrArArCrUrUrGr |
| | ArArArArArGrUrGrGrCrArCrCrGrArGrUrCrGrGrUrGrCrGrGrUrUrCrCrCrArUrUrGrArArUrU |
| | rCrArGrUrCrAmC*mA*mU*mA*mA*mA*mG*mC |
| SEQ ID NO: | mU*mA*rCrArGrCrUrUrUrArUrGrUrGrArCrUrArArUrGrUrUrUrUrArGrArGrCrUrArGrArAr |
| 145 | ArUrArGrCrArArGrUrUrArArArArUrArArGrGrCrUrArGrUrCrCrGrUrUrArUrCrArArCrUrUrGr |
| | ArArArArArGrUrGrGrCrArCrCrGrArGrUrCrGrGrUrGrCrGrGrUrUrCrCrCrArUrUrGrArArUrU |
| | rCrArGrUrCrAmCmA*mU*mA*mA*mA*mG*mC |
| SEQ ID NO: | mU*mA*rCrArGrCrUrUrUrArUrGrUrGrArCrUrArArUrGrUrUrUrUrArGrArGrCrUrArGrArAr |
| 146 | ArUrArGrCrArArGrUrUrArArArArUrArArGrGrCrUrArGrUrCrCrGrUrUrArUrCrArArCrUrUrGr |
| | ArArArArArGrUrGrGrCrArCrCrGrArGrUrCrGrGrUrGrCrGrGrUrUrCrCrCrArUrUrGrArArUrU |
| | rCrArGrUrCrAmCmAmU*mA*mA*mA*mG*mC |
| SEQ ID NO: | mU*mA*rCrArGrCrUrUrUrArUrGrUrGrArCrUrArArUrGrUrUrUrUrArGrArGrCrUrArGrArAr |
| 147 | ArUrArGrCrArArGrUrUrArArArArUrArArGrGrCrUrArGrUrCrCrGrUrUrArUrCrArArCrUrUrGr |
| | ArArArArArGrUrGrGrCrArCrCrGrArGrUrCrGrGrUrGrCrGrGrUrUrCrCrCrArUrUrGrArArUrU |
| | rCrArGrUrCrAmCmAmUmA*mA*mA*mG*mC |
| SEQ ID NO: | mU*mA*rCrArGrCrUrUrUrArUrGrUrGrArCrUrArArUrGrUrUrUrUrArGrArGrCrUrArGrA |
| 148 | rArArUrArGrCrArArGrUrUrArArArArUrArArGrGrCrUrArGrUrCrCrGrUrUrArUrCrArArCr |
| | UrUrGrArArArArArGrUrGrGrCrArCrCrGrArGrUrCrGrGrUrGrCrGrGrUrUrCrCrCrArUrUr |
| | GrArArUrUrCrArGrUrCrAmCmAmUmAmA*mA*mG*mC |
| SEQ ID NO: | mU*mA*rCrArGrCrUrUrUrArUrGrUrGrArCrUrArArUrGrUrUrUrUrArGrArGrCrUrArGrA |
| 149 | rArArUrArGrCrArArGrUrUrArArArArUrArArGrGrCrUrArGrUrCrCrGrUrUrArUrCrArArCr |
| | UrUrGrArArArArArGrUrGrGrCrArCrCrGrArGrUrCrGrGrUrGrCrGrGrUrUrCrCrCrArUrUr |
| | GrArArUrUrCrArGrUrCrAmCmAmUmAmAmA*mG*mC |
| SEQ ID NO: | mU*mA*rCrArGrCrUrUrUrArUrGrUrGrArCrUrArArUrGrUrUrUrUrArGrArGrCrUrArGrA |
| 150 | rArArUrArGrCrArArGrUrUrArArArArUrArArGrGrCrUrArGrUrCrCrGrUrUrArUrCrArArCr |
| | UrUrGrArArArArArGrUrGrGrCrArCrCrGrArGrUrCrGrGrUrGrCrGrGrUrUrCrCrCrArUrUr |
| | GrArArUrUrCrArGrUrCrAmCmAmUmAmAmAmG*mC |
| SEQ ID NO: | mU*mA*mC*rArGrCrUrUrUrArUrGrUrGrArCrUrArArUrGrUrUrUrUrArGrArGrCrUrArG |
| 151 | rArArArUrArGrCrArArGrUrUrArArArArUrArArGrGrCrUrArGrUrCrCrGrUrUrArUrCrArAr |
| | CrUrUrGrArArArArArGrUrGrGrCrArCrCrGrArGrUrCrGrGrUrGrCrGrGrUrUrCrCrCrArUr |
| | UrGrArArUrUrCrArGrUrCrArCrArUrAmA*mA*mG*rC |

(r = RNA base, m = 2′-O-methyl base, * = phosphorothioate bond)

TABLE 2
Prime Editing EcoRI insertion facilitated by chemically
synthesized PEG-RNAs targeting multiple HPRT loci using
plasmid-based prime editor protein (Cas9 H840A)

| | % | Std |
|---|---|---|
| SEQ_ID | cleavage | Dev |
| SEQ ID NO: | 21.7 | 0.4 |
| 1 | | |
| SEQ ID NO: | 8.2 | 0.3 |
| 2 | | |
| SEQ ID NO: | 1.0 | 0.3 |
| 3 | | |
| SEQ ID NO: | 6.1 | 0.3 |
| 4 | | |
| SEQ ID NO: | 1.4 | 0.1 |
| 5 | | |
| SEQ ID NO: | 3.4 | 0.3 |
| 6 | | |
| SEQ ID NO: | 1.7 | 0.1 |
| 7 | | |
| SEQ ID NO: | 4.6 | 0.1 |
| 8 | | |
| SEQ ID NO: | 2.8 | 0.0 |
| 9 | | |
| SEQ ID NO: | 10.2 | 0.0 |
| 10 | | |
| SEQ ID NO: | 8.1 | 0.1 |
| 11 | | |
| SEQ ID NO: | 7.3 | 0.3 |
| 12 | | |
| SEQ ID NO: | 8.9 | 0.3 |
| 13 | | |
| SEQ ID NO: | 0.5 | 0.4 |
| 14 | | |
| SEQ ID NO: | 0.1 | 0.2 |
| 15 | | |
| SEQ ID NO: | 0.2 | 0.3 |
| 16 | | |
| SEQ ID NO: | 0.8 | 0.2 |
| 17 | | |
| SEQ ID NO: | 1.7 | 0.0 |
| 18 | | |
| SEQ ID NO: | 1.7 | 0.1 |
| 19 | | |
| SEQ ID NO: | 2.0 | 0.2 |
| 20 | | |
| SEQ ID NO: | 1.8 | 0.1 |
| 21 | | |
| SEQ ID NO: | 6.1 | 0.3 |
| 22 | | |
| SEQ ID NO: | 3.7 | 0.2 |
| 23 | | |
| SEQ ID NO: | 7.3 | 0.3 |
| 24 | | |
| SEQ ID NO: | 4.3 | 0.2 |
| 25 | | |
| SEQ ID NO: | 0.0 | 0.0 |
| 26 | | |
| SEQ ID NO: | 0.0 | 0.0 |
| 27 | | |
| SEQ ID NO: | 0.0 | 0.0 |
| 28 | | |
| SEQ ID NO: | 0.0 | 0.0 |
| 29 | | |
| SEQ ID NO: | 0.0 | 0.0 |
| 30 | | |
| SEQ ID NO: | 0.0 | 0.0 |
| 31 | | |
| SEQ ID NO: | 0.0 | 0.0 |
| 32 | | |
| SEQ ID NO: | 0.3 | 0.2 |
| 33 | | |
| SEQ ID NO: | 0.0 | 0.0 |
| 34 | | |
| SEQ ID NO: | 0.0 | 0.0 |
| 35 | | |
| SEQ ID NO: | 0.0 | 0.0 |
| 36 | | |
| SEQ ID NO: | 0.0 | 0.0 |
| 37 | | |
| SEQ ID NO: | 0.0 | 0.0 |
| 38 | | |
| SEQ ID NO: | 0.5 | 0.0 |
| 39 | | |
| SEQ ID NO: | 0.4 | 0.1 |
| 40 | | |
| SEQ ID NO: | 0.4 | 0.0 |
| 41 | | |
| SEQ ID NO: | 11.6 | 0.2 |
| 42 | | |
| SEQ ID NO: | 10.6 | 0.6 |
| 43 | | |
| SEQ ID NO: | 10.2 | 0.3 |
| 44 | | |
| SEQ ID NO: | 13.2 | 0.6 |
| 45 | | |
| SEQ ID NO: | 11.8 | 0.4 |
| 46 | | |
| SEQ ID NO: | 6.6 | 0.2 |
| 47 | | |
| SEQ ID NO: | 15.1 | 0.7 |
| 48 | | |
| SEQ ID NO: | 13.4 | 0.4 |
| 49 | | |
| SEQ ID NO: | 1.3 | 0.2 |
| 50 | | |
| SEQ ID NO: | 0.7 | 0.1 |
| 51 | | |
| SEQ ID NO: | 0.9 | 0.1 |
| 52 | | |
| SEQ ID NO: | 6.8 | 0.1 |
| 53 | | |
| SEQ ID NO: | 9.6 | 0.4 |
| 54 | | |
| SEQ ID NO: | 10.6 | 0.7 |
| 55 | | |
| SEQ ID NO: | 11.9 | 0.5 |
| 56 | | |
| SEQ ID NO: | 11.4 | 0.6 |
| 57 | | |
| SEQ ID NO: | 15.7 | 0.8 |
| 58 | | |
| SEQ ID NO: | 8.6 | 0.0 |
| 59 | | |
| SEQ ID NO: | 15.1 | 0.5 |
| 60 | | |
| SEQ ID NO: | 2.0 | 0.2 |
| 61 | | |
| SEQ ID NO: | 2.0 | 0.1 |
| 62 | | |
| SEQ ID NO: | 2.2 | 0.3 |
| 63 | | |
| SEQ ID NO: | 4.1 | 0.2 |
| 64 | | |
| SEQ ID NO: | 5.8 | 1.8 |
| 65 | | |
| SEQ ID NO: | 6.5 | 0.2 |
| 66 | | |
| SEQ ID NO: | 6.5 | 0.5 |
| 67 | | |
| SEQ ID NO: | 3.4 | 1.0 |
| 68 | | |
| SEQ ID NO: | 6.8 | 0.4 |
| 69 | | |
| SEQ ID NO: | 4.8 | 0.2 |
| 70 | | |
| SEQ ID NO: | 0.8 | 0.1 |
| 71 | | |
| SEQ ID NO: | 1.9 | 0.1 |
| 72 | | |
| SEQ ID NO: | 6.5 | 0.3 |
| 73 | | |
| SEQ ID NO: | 13.3 | 0.6 |
| 74 | | |
| SEQ ID NO: | 13.9 | 0.1 |
| 75 | | |
| SEQ ID NO: | 13.9 | 0.3 |
| 76 | | |
| SEQ ID NO: | 16.4 | 1.1 |
| 77 | | |
| SEQ ID NO: | 13.9 | 0.5 |
| 78 | | |
| SEQ ID NO: | 12.5 | 0.4 |
| 79 | | |
| SEQ ID NO: | 11.1 | 0.2 |
| 80 | | |
| SEQ ID NO: | 6.9 | 0.5 |
| 81 | | |
| SEQ ID NO: | 0.8 | 0.2 |
| 82 | | |
| SEQ ID NO: | 0.6 | 0.1 |
| 83 | | |
| SEQ ID NO: | 0.7 | 0.1 |
| 84 | | |
| SEQ ID NO: | 0.9 | 0.1 |
| 85 | | |
| SEQ ID NO: | 0.5 | 0.0 |
| 86 | | |
| SEQ ID NO: | 0.6 | 0.0 |
| 87 | | |
| SEQ ID NO: | 0.3 | 0.0 |
| 88 | | |
| SEQ ID NO: | 0.3 | 0.1 |
| 89 | | |
| SEQ ID NO: | 1.4 | 0.2 |
| 90 | | |
| SEQ ID NO: | 0.7 | 0.1 |
| 91 | | |
| SEQ ID NO: | 10.4 | 0.3 |
| 92 | | |
| SEQ ID NO: | 11.0 | 0.3 |
| 93 | | |
| SEQ ID NO: | 7.4 | 0.2 |
| 94 | | |
| SEQ ID NO: | 7.1 | 0.2 |
| 95 | | |
| SEQ ID NO: | 6.8 | 0.4 |
| 96 | | |
| SEQ ID NO: | 5.8 | 0.1 |
| 97 | | |
| SEQ ID NO: | 6.1 | 0.4 |
| 98 | | |
| SEQ ID NO: | 3.6 | 0.2 |
| 99 | | |
| SEQ ID NO: | 3.7 | 0.3 |
| 100 | | |
| SEQ ID NO: | 2.6 | 0.1 |
| 101 | | |
| SEQ ID NO: | 8.4 | 0.1 |
| 102 | | |
| SEQ ID NO: | 10.0 | 0.3 |
| 103 | | |
| SEQ ID NO: | 6.9 | 0.3 |
| 104 | | |
| SEQ ID NO: | 7.6 | 0.1 |
| 105 | | |
| SEQ ID NO: | 8.9 | 0.6 |
| 106 | | |
| SEQ ID NO: | 8.4 | 0.0 |
| 107 | | |
| SEQ ID NO: | 3.1 | 0.1 |
| 108 | | |
| SEQ ID NO: | 1.9 | 0.2 |
| 109 | | |
| SEQ ID NO: | 2.3 | 0.2 |
| 110 | | |
| SEQ ID NO: | 0.9 | 0.2 |
| 111 | | |
| SEQ ID NO: | 5.8 | 0.2 |
| 112 | | |
| SEQ ID NO: | 1.9 | 0.4 |
| 113 | | |
| SEQ ID NO: | 10.1 | 0.7 |
| 114 | | |
| SEQ ID NO: | 5.5 | 0.3 |
| 115 | | |
| SEQ ID NO: | 22.0 | 0.4 |
| 116 | | |
| SEQ ID NO: | 0.9 | 0.0 |
| 117 | | |
| SEQ ID NO: | 2.6 | 0.1 |
| 118 | | |
| SEQ ID NO: | 16.1 | 0.2 |
| 119 | | |
| SEQ ID NO: | 0.4 | 0.2 |
| 120 | | |
| SEQ ID NO: | 0.0 | 0.0 |
| 121 | | |
| SEQ ID NO: | 0.1 | 0.1 |
| 122 | | |
| SEQ ID NO: | 0.6 | 0.0 |
| 123 | | |
| SEQ ID NO: | 13.8 | 0.6 |
| 124 | | |
| SEQ ID NO: | 16.4 | 0.2 |
| 125 | | |
| SEQ ID NO: | 9.9 | 0.9 |
| 126 | | |
| SEQ ID NO: | 6.3 | 0.2 |
| 127 | | |
| SEQ ID NO: | 21.1 | 0.2 |
| 128 | | |
| SEQ ID NO: | 1.2 | 0.1 |
| 129 | | |
| SEQ ID NO: | 2.5 | 0.3 |
| 130 | | |
| SEQ ID NO: | 17.4 | 0.3 |
| 131 | | |
| SEQ ID NO: | 0.6 | 0.2 |
| 132 | | |
| SEQ ID NO: | 0.5 | 0.1 |
| 133 | | |
| SEQ ID NO: | 0.1 | 0.2 |
| 134 | | |
| SEQ ID NO: | 0.8 | 0.1 |
| 135 | | |
| SEQ ID NO: | 15.9 | 0.4 |
| 136 | | |
| SEQ ID NO: | 20.4 | 0.4 |
| 137 | | |
| SEQ ID NO: | 0.7 | 0.1 |
| 138 | | |
| SEQ ID NO: | 0.7 | 0.1 |
| 139 | | |
| SEQ ID NO: | 0.5 | 0.0 |
| 140 | | |
| SEQ ID NO: | 0.5 | 0.0 |
| 141 | | |
| SEQ ID NO: | 0.7 | 0.1 |
| 142 | | |
| SEQ ID NO: | 0.5 | 0.1 |
| 143 | | |
| SEQ ID NO: | 19.6 | 0.9 |
| 144 | | |
| SEQ ID NO: | 20.0 | 0.9 |
| 145 | | |
| SEQ ID NO: | 19.5 | 1.0 |
| 146 | | |
| SEQ ID NO: | 20.2 | 1.0 |
| 147 | | |
| SEQ ID NO: | 19.3 | 0.9 |
| 148 | | |
| SEQ ID NO: | 19.5 | 0.5 |
| 149 | | |
| SEQ ID NO: | 18.9 | 0.9 |
| 150 | | |

### Example 2. Representative Modification Patterns Show Greatly Improved Efficacy Over the Standard Modification Pattern

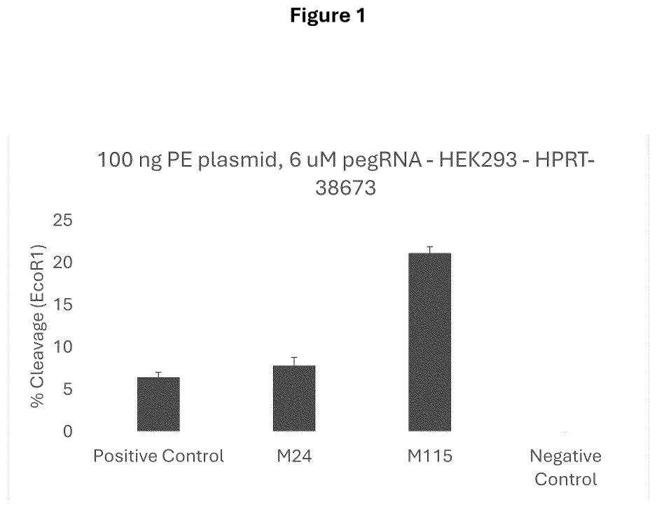

Referring to FIG. 1, targeting the HPRT-38673 locus, PEG RNAs were designed to insert an EcoR1 site (GAATTC) and tested specific PS bonds and 2′-O-Methylation at the 5′ and 3′ termini compared to the Standard Modification pattern as disclosed in Hendel et al. (“Positive Control”). Plasmid expressing prime editor protein and chemically synthesized PEG RNAs were introduced into HEK293 cells by electroporation (Lonza Nucelofector) and genomic DNA was extracted after 72 hr. Insertion of the EcoRI sequence was assessed by restriction digest and DNA analysis on the Fragment Analyzer (Agilent). A specific pattern of PS bonds and 2′-O-methylation outperforms the Standard Modification pattern (“Positive Control”). The positive control was SEQ ID No: 151, M24 is SEQ ID No: 73, and M115 is SEQ ID No: 137. Negative Control=cell only.

While the invention has been described in detail and with reference to specific examples thereof, it will be apparent to one skilled in the art that various changes and modifications can be made therein without departing from the spirit and scope thereof.

What is claimed is:

1. A modified prime editing guide RNA (“mpegRNA”) nucleotide sequence comprising:
i. one or more modified nucleotides at the 5′ end;
ii. one or more modified nucleotides at the 3′ end; or
iii. one or more modified nucleotides at both the 5′ and 3′ ends,
wherein the modified nucleotides comprise a modification selected from the group consisting of 2′-O-methyl modification, phosphorothioate linkage modification, and combinations thereof.

2. The mpegRNA nucleotide sequence of claim 1, wherein at least two, three, four, five, six, seven, eight, nine, or ten of the nucleotides at the 5′ end of the mpegRNA are modified nucleotides.

3. The mpegRNA nucleotide sequence of any one of claims 1-2, comprising modified nucleotides at the 5′ end, wherein the modifications are 3 nucleotides with 2′-O-methyl modification, and 1 nucleotide with a phosphorothioate linkage modification.

4. The mpegRNA nucleotide sequence of any one of claims 1-3, comprising modified nucleotides at the 5′ end, wherein the modifications are 2 nucleotides with 2′-O-methyl modification, and 2 nucleotides with a phosphorothioate linkage modification.

5. The mpegRNA nucleotide sequence of any one of claims 1-4, wherein at least two, three, four, five, six, seven, eight, nine, or ten of the nucleotides at the 3′ end are modified nucleotides.

6. The mpegRNA nucleotide sequence of any one of claims 1-5, comprising modified nucleotides at the 3′ end, wherein the modifications are 7 nucleotides with 2′-O-methyl modifications and 7 nucleotides with phosphorothioate linkage modifications.

7. The mpegRNA nucleotide sequence of any one of claims 1-6, comprising modified nucleotides at the 3′ end, wherein the modifications are 6 nucleotides with 2′-O-methyl modifications and 1 nucleotide with phosphorothioate linkage modifications.

8. The mpegRNA nucleotide sequence of any one of claims 1-7, comprising modified nucleotides at the 3′ end, wherein the modifications are 5 nucleotides with 2′-O-methyl modifications and 1 nucleotide with phosphorothioate linkage modifications.

9. The mpegRNA nucleotide sequence of any one of claims 1-8, comprising selected from the group consisting of:
i) a nucleotide sequence comprising modified nucleotides at the 5′ end, wherein the modifications are 3 nucleotides with 2′-O-methyl modification, and 1 nucleotide with a phosphorothioate linkage modification;
ii) a nucleotide sequence comprising modified nucleotides at the 5′ end, wherein the modifications are 2 nucleotides with 2′-O-methyl modification, and 2 nucleotides with a phosphorothioate linkage modification;
iii) a nucleotide sequence comprising modified nucleotides at the 3′ end, wherein the modifications are 7 nucleotides with 2′-O-methyl modifications and 7 nucleotides with phosphorothioate linkage modifications;
iv) a nucleotide sequence comprising modified nucleotides at the 3′ end, wherein the modifications are 6 nucleotides with 2′-O-methyl modifications and 1 nucleotide with phosphorothioate linkage modifications;
v) a nucleotide sequence comprising modified nucleotides at the 3′ end, wherein the modifications are 5 nucleotides with 2′-O-methyl modifications and 1 nucleotide with phosphorothioate linkage modifications; and
vi) any combinations thereof.

10. A modified prime editing guide RNA (“mpegRNA”) comprising:
i. a targeting sequence comprising one or more modified nucleotides at the 5′ end; and
ii. a primer binding site comprising one or more modified nucleotides at the 3′ end,
wherein the one or more modified nucleotides comprise at least one modification selected from the group consisting of 2′-O-methyl modification, phosphorothioate linkage modification, and combinations thereof.

11. The mpegRNA nucleotide sequence of claim 10 wherein the targeting sequence comprises at least two, three, four, five, six, seven, eight, nine, or ten modified nucleotides.

12. The mpegRNA nucleotide sequence of any one of claims 10-11, wherein the targeting sequence comprises three nucleotides with 2′-O-methyl modification, and one nucleotide with a phosphorothioate linkage modification.

13. The mpegRNA nucleotide sequence of any one of claims 10-12, wherein the targeting sequence comprises two nucleotides with 2′-O-methyl modification, and two nucleotides with a phosphorothioate linkage modification.

14. The mpegRNA nucleotide sequence of any one of claims 10-13, wherein the primer binding site comprises at least two, three, four, five, six, seven, eight, nine, or ten modified nucleotides.

15. The mpegRNA nucleotide sequence of any one of claims 10-14, wherein the primer binding site comprises seven nucleotides with 2′-O-methyl modifications and seven nucleotides with phosphorothioate linkage modifications.

16. The mpegRNA nucleotide sequence of any one of claims 10-15, wherein the primer binding site comprises six nucleotides with 2′-O-methyl modifications and one nucleotide with phosphorothioate linkage modifications.

17. The mpegRNA nucleotide sequence of any one of claims 10-16, wherein the primer binding site comprises five nucleotides with 2′-O-methyl modifications and one nucleotide with phosphorothioate linkage modifications.

18. The mpegRNA nucleotide sequence of any one of claims 10-17, comprising selected from the group consisting of:
vii) a targeting sequence comprising three nucleotides with 2′-O-methyl modifications and one nucleotide with a phosphorothioate linkage modification;
viii) a targeting sequence comprising two nucleotides with 2′-O-methyl modifications and two nucleotides with a phosphorothioate linkage modifications;
ix) a primer binding site comprising seven nucleotides with 2′-O-methyl modifications and seven nucleotides with phosphorothioate linkage modifications;
x) a primer binding site comprising six nucleotides with 2′-O-methyl modifications and one nucleotide with phosphorothioate linkage modifications;
xi) a primer binding site comprising five nucleotides with 2′-O-methyl modifications and one nucleotide with phosphorothioate linkage modification; and
xii) any combinations thereof.

19. The mpegRNA nucleotide sequence of any one of claims 1-18, wherein the mpegRNA comprises five nucleotides with 2′-O-methyl modifications and one nucleotide with phosphorothioate linkage modifications at the 3′ end, and wherein the mpegRNA comprises two nucleotides with 2′-O-methyl modifications and two nucleotide with phosphorothioate linkage modifications at the 5′ end.

20. The mpegRNA nucleotide sequence of any one of claims 1-18, wherein the mpegRNA comprises six nucleotides with 2′-O-methyl modifications and one nucleotide with phosphorothioate linkage modifications at the 3′ end, and wherein the mpegRNA comprises two nucleotides with 2′-O-methyl modifications and two nucleotide with phosphorothioate linkage modifications at the 5′ end.

21. The mpegRNA of any one of claims 1-20, wherein off-target editing relative to an unmodified gRNA is reduced by an amount selected from the group consisting of 80%, 85%, 90%, 95%, and 99%.

22. The mpegRNA of any one of claims 1-21, wherein the at least one modified nucleotide alters base-pairing thermostability.

23. The mpegRNA of any one of claims 1-22, wherein said at least one modified nucleotide enhances base-pairing thermostability.

24. The mpegRNA RNA of any one of claims 1-23, wherein said at least one modified nucleotide decreases base-pairing thermostability.

25. The mpegRNA of any one of claims 1-24, wherein the at least one modified nucleotide is a specificity-altering modification.

26. The mpegRNA of any one of claims 1-25, wherein the modified mpegRNA is chemically synthesized.

27. A set or library of RNA molecules comprising two or more mpegRNAs of any one of claims 1-26.

28. A kit comprising the mpegRNA of any one of claims 1-26.

29. An array of RNA molecules comprising two or more mpegRNAs of any one of claims 1-26.

30. A method for inducing gene regulation of a target nucleic acid in a cell, the method comprising:
introducing into the cell:
(a) an mpegRNA of any one of claims 1-26;
(b) a Cas9 polypeptide, an mRNA encoding a Cas9 polypeptide, or a recombinant expression vector comprising a nucleotide sequence encoding a Cas9 polypeptide,
wherein the mpegRNA guides the Cas9 polypeptide to the target nucleic acid, and wherein the mpegRNA induces a gene regulation of the target nucleic acid with an enhanced activity relative to a corresponding unmodified mpegRNA.

31. The method of claim 30, wherein the modified mpegRNA in (a) and the Cas9 polypeptide in (b) are introduced into the cell in a ribonucleoprotein (RNP) complex.

32. The method of any one of claims 30-31, wherein the gene regulation induced by the introduction of (a) and (b) is stable in the cell for at least 24 hours.

33. The method of any one of claims 30-32, wherein the mpegRNA in (a) and the Cas9 polypeptide in (b) are introduced into the cell in a lipofection reagent.

34. The method of any one of claims 30-33, wherein the mpegRNA in (a) and the Cas9 polypeptide in (b) are introduced into the cell via exosomes.

35. The method of any one of claims 30-34, wherein the mpegRNA in (a) and the Cas9 polypeptide in (b) are introduced into the cell via lipid nanoparticles.

36. The method of any one of claims 30-35, wherein the mpegRNA in (a) and the Cas9 polypeptide in (b) are introduced into the cell via viral vector.
